(19) United States
(12) Patent Application Publication
(10) Pub. No.: US 2018/0355001 A1
(43) Pub. Date: Dec. 13, 2018

(54) PRODUCTION OF TERPENES, TERPENOIDS, AND DERIVATIVES THEREOF IN RECOMBINANT HOSTS

(71) Applicant: Evolva SA, Reinach (CH)

(72) Inventors: Thomas Tange, Riehen (CH); Jens Klein, Oberwil (CH); Federico Brianza, Riehen (CH)

(21) Appl. No.: 15/778,804

(22) PCT Filed: Nov. 25, 2016
(86) PCT No.: PCT/EP2016/078903

§ 371 Date: May 24, 2018

Related U.S. Application Data
(60) Provisional application No. 62/260,456, filed on Nov. 27, 2015.

(51) Int. Cl. C07K 14/435; C12N 15/62; C12N 9/88; C12N 15/81

(52) U.S. Cl. CPC C07K 14/435; C12N 15/62; C12Y 402/03009; C12N 15/81; C12N 9/88

(57) ABSTRACT

The invention relates to recombinant microorganisms and methods for producing terpene compounds, terpenoid compounds, and precursors thereof derived from (2Z,6E)-farnesyl diphosphate.

### BACKGROUND OF THE INVENTION

### Field of the Invention

This disclosure relates to recombinant production of terpenes, terpenoids, and precursors thereof in recombinant hosts. In particular, this disclosure relates to production of terpenes and precursors of terpenes comprising (2Z,6E)-farnesyl diphosphate ((2Z,6E)-FPP), α-cedrene, prezizaene, α-acoradiene, β-curcumene, (Z)-nerolidol, α-bisabolol, or (2Z,6E)-farnesol in recombinant hosts.

### Description of Related Art

Terpenes and the related terpenoids comprise a large class of biologically derived organic molecules. Terpenes and terpenoids are derived from five-carbon isoprene units and are accordingly also referred to as isoprenoids. They are produced from isoprenoid pyrophosphates (IPPs) which are organic molecules that serve as precursors in the biosynthesis of a number of biologically and commercially important molecules.

Terpenoids can be found in all classes of living organisms, and comprises the largest group of natural products. Plant terpenoids are used extensively for their aromatic qualities and play a role in traditional herbal remedies and are under investigation for antibacterial, antineoplastic, and other pharmaceutical functions. Terpenoids contribute to the scent of eucalyptus, the flavors of cinnamon, cloves, and ginger, and the color of yellow flowers. Well-known terpenoids include citral, menthol, camphor, Salvinorin A in the plant Salvia divinorum, and cannabinoids.

While the biosynthetic steps leading from isopentenylpyrophosphate (IPP) and/or dimethylallylpyrophosphate (DMAPP) to terpenoids are universal, two different pathways leading to IPP and DMAPP exist—the mevalonic acid pathway and the non-mevalonic, 2-C-methyl-D-erythritol 4-phosphate/1-deoxy-D-xylulose 5-phosphate (MEP/DOXP) pathway. The mevalonate pathway is responsible for the production of isoprenoid-derived molecules in numerous organisms.

The part of the mevalonate pathway that generates the basic C5 isoprenoid pyrophosphates, isopentenyl pyrophosphate (IPP) and dimethylallyl pyrophosphate (DMAPP), comprises seven enzymatic steps. The seven S. cerevisiae genes involved in these steps are (in consecutive order in the pathway): ERG10, ERG13, HMGR, ERG12, ERG8, ERG19 and IDI1. IPP and DMAPP are the isoprene units that form the basis for synthesis of higher order isoprenoid pyrophosphate precursors containing any number of isoprene units between two and ten. The most important ones are geranyl pyrophosphate (GPP), farnesyl pyrophosphate (FPP) and geranylgeranyl pyrophosphate (GGPP).

The isoprenoid pyrophosphate precursor FPP can be converted to terpenes or terpenoids through either a transoid or cisoid pathway. The pathway utilized, and the terpene or terpenoid ultimately synthesized, depends on the conformation of the FPP and the ability of the enzyme to show transoid and/or cisoid catalytic activity. Generally, (2E,6E)-FPP is precursor to transoid products whereas (2Z,6E)-FPP and (2Z,6Z)-FPP are precursors to cisoid products. (2Z,6E)-FPP occurs naturally in very low levels relative to (2E,6E)-FPP—only 3% to 14% in an in vitro experiment, depending on the origin of the FPP synthase used. See Thulasiram and Poulter, 2006, J. Am. Chem. Soc. 238(49):15819-23. (2Z,6Z)-FPP has been shown to exist in certain organisms. See Sallaud et al., 2009, Plant Cell 21(1):301-17. The synthesis of cisoid terpenes and terpenoids can be catalyzed by terpene synthases selective for use of (2Z,6E)-FPP and/or (2Z,6Z)-FPP as a substrate, or by terpene synthases that are additionally capable of catalyzing the synthesis of transoid terpenes and terpenoids from a (2E,6E)-FPP substrate.

Many isoprenoid molecules have high commercial value. As recovery and purification of isoprenoid molecules have proven to be labor intensive and inefficient, there remains a need for a recombinant production system that can accumulate high yields of desired isoprenoid molecules. Such a production system is highly desirable for both economical and sustainability reasons.

### SUMMARY OF THE INVENTION

It is against the above background that the present invention provides certain advantages and advancements over the prior art.

Although this invention as disclosed herein is not limited to specific advantages or functionality, the invention disclosed herein provides a recombinant host comprising a gene encoding a heterologous (2Z,6E)-farnesyl diphosphate synthase ((2Z,6E)-FPPS) polypeptide; wherein the host is capable of producing a (2Z,6E)-farnesyl diphosphate ((2Z,6E)-FPP) compound and/or a compound derived or produced from (2Z,6E)-FPP.

In some aspects of the recombinant host or methods disclosed herein, the recombinant host comprises a gene encoding the (2Z,6E)-FPPS polypeptide that encodes an amino acid sequence having 70% or greater identity to the amino acid sequence set forth in SEQ ID NO:4.

In some aspects of the recombinant host or methods disclosed herein, the recombinant host further comprises a gene encoding a terpene synthase polypeptide, wherein (2Z,6E)-FPP is a substrate for said terpene synthase.

In some aspects, (2Z,6E)-FPP and (2E,6E)-farnesyl diphosphate ((2E,6E)-FPP) are substrates for said terpene synthase.

In some aspects, the terpene synthase is a 4,5-di-epi-aristolochene synthase (TEAS).

In some aspects, the gene encoding the TEAS polypeptide encodes an amino acid sequence having 70% or greater identity to the amino acid sequence set forth in SEQ ID NO:6.

The invention further provides a recombinant host comprising:
(a) a gene encoding a (2Z,6E)-FPPS polypeptide having 70% or greater identity to an amino acid sequence set forth in SEQ ID NO:4; and
(b) a gene encoding a TEAS polypeptide having 70% or greater identity to an amino acid sequence set forth in SEQ ID NO:6;
wherein at least one of said genes is a heterologous gene.

In some aspects of the recombinant host or methods disclosed herein, the recombinant host is engineered to have reduced expression of an endogenous gene encoding:
(a) a (2E,6E)-FPPS polypeptide;
(b) a geranyl diphosphate synthase (GPPS) polypeptide; or
(c) a polypeptide having both (2E,6E)-FPPS and GPPS enzymatic activity.

In some aspects of the recombinant host or methods disclosed herein, the endogenous gene encoding a (2E,6E)-FPPS polypeptide is ERG20.

In some aspects, the ERG20 gene encodes a polypeptide having 70% or greater identity to an amino acid sequence set forth in SEQ ID NO:2.

In some aspects of the recombinant host or methods disclosed herein, the recombinant host produces the compound (2Z,6E)-FPP.

The invention further provides a method of producing (2Z,6E)-FPP comprising:
(a) growing a recombinant host in a culture medium, under conditions in which the genes are expressed, wherein (2Z,6E)-FPP is synthesized by the recombinant host; and
(b) isolating (2Z,6E)-FPP.

In some aspects of the methods disclosed herein, the (2Z,6E)-FPPS polypeptide comprises a (2Z,6E)-FPPS polypeptide having 70% or greater identity to the amino acid sequence set forth in SEQ ID NO: 4.

The invention further provides a method of producing a terpene or terpenoid derived from (2Z,6E)-FPP comprising:
(a) growing a recombinant host in a culture medium, under conditions in which the genes are expressed, wherein the terpene or terpenoid is synthesized by the recombinant host converting (2Z,6E)-FPP to said terpene or terpenoid; and
(b) isolating the terpene or terpenoid derived from (2Z,6E)-FPP.

In some aspects of the recombinant host or methods disclosed herein, the host produces a terpene or terpenoid compound derived from (2Z,6E)-FPP, the compound comprising α-cedrene, prezizaene, α-acoradiene, β-curcumene, (Z)-Nerolidol, α-bisabolol, and/or (2Z,6E)-farnesol.

In some aspects of the methods disclosed herein, the conversion of (2Z,6E)-FPP to a terpene or terpenoid is catalyzed by a terpene synthase polypeptide, wherein (2Z,6E)-FPP is a substrate for said terpene synthase.

In some aspects of the methods disclosed herein, the terpene synthase is a TEAS.

In some aspects of the methods disclosed herein, the TEAS polypeptide encodes an amino acid sequence having 70% or greater identity to the amino acid sequence set forth in SEQ ID NO:6.

In some aspects, the methods disclosed herein further comprise a step of modifying the terpene or terpenoid.

In some aspects, the terpene or terpenoid is oxygenated.

In some aspects, oxygenation of the terpene or terpenoid is catalyzed by a cytochrome P450 polypeptide.

In some aspects, the terpene or terpenoid is methylated.

In some aspects, a sulfonate group is added to the terpene or terpenoid.

In some aspects, a halogen is added to the terpene or terpenoid.

In some aspects of the recombinant host or methods disclosed herein, the recombinant host comprises a microorganism that is a plant cell, a mammalian cell, an insect cell, a fungal cell, or a bacterial cell.

In some aspects of the recombinant host or methods disclosed herein, the bacterial cell comprises Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacteria cells.

In some aspects of the recombinant host or methods disclosed herein, the fungal cell comprises a yeast cell.

In some aspects of the recombinant host or methods disclosed herein, the yeast cell comprises a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

In some aspects the yeast cell comprises a Saccharomycete.

In some aspects, the yeast cell comprises a cell from the Saccharomyces cerevisiae species.

The invention further provides a cell culture broth comprising:
(a) the recombinant host disclosed herein; and
(b) (2Z,6E)-FPP, α-cedrene, prezizaene, α-acoradiene, β-curcumene, (Z)-Nerolidol, α-bisabolol, and/or (2Z,6E)-farnesol produced by the recombinant host disclosed herein;
wherein (2Z,6E)-FPP, α-cedrene, prezizaene, α-acoradiene, β-curcumene, (Z)-Nerolidol, α-bisabolol, and/or (2Z,6E)-farnesol is present at a concentration of at least 0.1 mg/liter of the culture broth.

In some aspects, the cell culture broth has an increased level of the metabolite (2Z,6E)-farnesol relative to a cell culture broth comprising a corresponding host lacking the gene encoding a heterologous (2Z,6E)-FPPS.

The invention further provides a cell culture broth comprising (2Z,6E)-FPP, α-cedrene, prezizaene, α-acoradiene, β-curcumene, (Z)-Nerolidol, α-bisabolol, and/or (2Z,6E)-farnesol; wherein (2Z,6E)-FPP, α-cedrene, prezizaene, α-acoradiene, β-curcumene, (Z)-Nerolidol, α-bisabolol, and/or (2Z,6E)-farnesol is present at a concentration of at least 0.1 mg/liter of the culture broth, and is produced by culturing the cells of the recombinant host of any one of claims 1-18 in a culture media.

The invention further provides a cell lysate comprising (2Z,6E)-FPP, α-cedrene, prezizaene, α-acoradiene, β-curcumene, (Z)-Nerolidol, α-bisabolol, and/or (2Z,6E)-farnesol produced by the recombinant host disclosed herein.

The invention further provides a composition of terpenes and/or terpenoids comprising (2Z,6E)-FPP, α-cedrene, prezizaene, α-acoradiene, 3-curcumene, (Z)-Nerolidol, α-bisabolol, and/or (2Z,6E)-farnesol produced by the recombinant host disclosed herein, wherein the relative levels of terpenes and/or terpenoids in the composition correspond to the relative levels of terpene and/or terpenoid accumulation in the recombinant host.

In some aspects, the composition of terpenes and/or terpenoids has an increased level of the metabolite (2Z,6E)-farnesol relative to a composition of terpenes and/or terpenoids produced by a corresponding host lacking the gene encoding a heterologous (2Z,6E)-FPPS

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1A shows the gas chromatography/electron ionization-mass spectrometry (GC/EI-MS) chromatogram of the isopropyl myristate layer of the engineered S. cerevisiae cultures of Example 2 (top) and Example 4 (bottom). For each strain, 300 μL of the isopropyl myristate layer was sampled from the shake flask. A 10 μL aliquot of the organic phase was diluted 1:100 using ethyl acetate before GC/EI-MS analysis. GC/EI-MS analyses were carried out using an Agilent 7890C gas chromatograph coupled to a 5975C quadrupole mass selective detector (MSD) with inert ion course using electron ionization. The GC was equipped with an HB5 ms capillary column (30 m×0.25 mm, film thickness 0.25 μm). The EI system was set with an ionization energy of 70 eV. Helium was used as carrier gas at a flow rate of 1.0 mL/min. Injector and ion source temperatures were set to 250° C. The injection volume was 1 μL. Experiments were run in splitless mode. The oven temperature was programmed to hold 80° C. for 2 minutes, then increase 30° C./min to 160° C., hold for 0 minutes, then increase 3° C./min to 170° C., hold for 0 minutes, then increase 30° C./min to 300° C., and hold for 2 minutes. The overall run time was 14.333 min. Data was evaluated using ChemStation E.02.01.1177, NIST Mass Spectral Search Program for the NIST/EPA/NHI Mass Spectral Library Version 2.0 g, build Mai 19 2011, and MassFinder 4.25 software. Results were based on MS similarity. No retention index (RI) was applied.

FIG. 1B shows the relative proportions of the volatile components in the organic isopropyl myristate layer of the engineered S. cerevisiae cultures of Example 2 and Example 4, as detected by GC/EI-MS. Peak numbers given correspond to the peak labels of FIG. 1A.

FIG. 2 shows a biosynthetic route from IPP and/or DMAPP to 4,5-di-epi-aristolochene in a S. cerevisiae strain comprising and expressing genes encoding an endogenous ERG20 polypeptide (SEQ ID NO:1, SEQ ID NO:2) and a Nicotiana attenuata TEAS polypeptide (SEQ ID NO:5, SEQ ID NO:6), as described in Example 4 (left), and a biosynthetic route from IPP and/or DMAPP to cisoid terpenes, terpenoids, and precursors thereof in a S. cerevisiae strain comprising and expressing genes encoding an endogenous ERG20 polypeptide (SEQ ID NO:1, SEQ ID NO:2), a Mycobacterium tuberculosis (2Z,6E)-FPPS (SEQ ID NO:3, SEQ ID NO:4), and a Nicotiana attenuata TEAS polypeptide (SEQ ID NO:5, SEQ ID NO:6), as described in Example 2 (right).

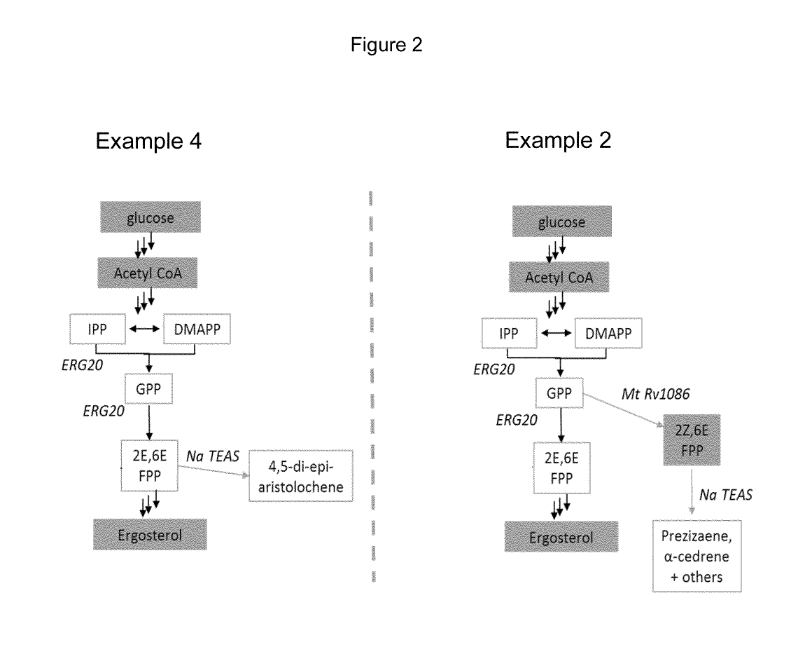

### DETAILED DESCRIPTION OF THE INVENTION

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the terms “increased level”, “decreased level” “significantly increased level”, “significantly decreased level”, and variants of these terms, are used herein to represent the inherent degree of uncertainty that can be attributed to any quantitative comparison, value, measurement, or other representation. These terms are also used herein to represent the degree by which a comparative value or other representation can vary from a stated reference without resulting in a change in the basic function of the subject matter at issue.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide”, “nucleotide”, “oligonucleotide”, and “nucleic acid” can be used interchangeably to refer to nucleic acid comprising DNA, RNA, derivatives thereof, or combinations thereof either in single-stranded or double-stranded form in context as understood by the skilled worker.

As used herein, the terms “microorganism,” “microorganism host,” “microorganism host cell,” “recombinant host,” and “recombinant host cell” can be used interchangeably. As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into a host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In some aspects, said recombinant genes are encoded by cDNA. In other embodiments, recombinant genes are synthetic and/or codon-optimized for expression in S. cerevisiae.

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a yeast gene. In some embodiments, the gene is endogenous to S. cerevisiae, including, but not limited to S. cerevisiae strain S288C. In some embodiments, an endogenous yeast gene is overexpressed. As used herein, the term “overexpress” is used to refer to the expression of a gene in an organism at levels higher than the level of gene expression in a wild type organism. See, e.g., Prelich, 2012, Genetics 190:841-54. In some embodiments, an endogenous yeast gene is deleted. See, e.g., Giaever & Nislow, 2014, Genetics 197(2):451-65. As used herein, the terms “deletion,” “deleted,” “knockout,” and “knocked out” can be used interchangeably to refer to an endogenous gene that has been manipulated to no longer be expressed in an organism, including, but not limited to, S. cerevisiae.

As used herein, the terms “heterologous sequence” and “heterologous coding sequence” are used to describe a sequence derived from a species other than the recombinant host. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

A “selectable marker” can be one of any number of genes that complement host cell auxotrophy, provide antibiotic resistance, or result in a color change. Linearized DNA fragments of the gene replacement vector then are introduced into the cells using methods well known in the art (see below). Integration of the linear fragments into the genome and the disruption of the gene can be determined based on the selection marker and can be verified by, for example, PCR or Southern blot analysis. Subsequent to its use in selection, a selectable marker can be removed from the genome of the host cell by, e.g., Cre-LoxP systems (see, e.g., Gossen et al., 2002, Ann. Rev. Genetics 36:153-173 and U.S. 2006/0014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene.

As used herein, the terms “variant” and “mutant” are used to describe a protein sequence that has been modified at one or more amino acids, compared to the wild-type sequence of a particular protein.

As used herein, the term “inactive fragment” is a fragment of the gene that encodes a protein having, e.g., less than about 10% (e.g., less than about 9%, less than about 8%, less than about 7%, less than about 6%, less than about 5%, less than about 4%, less than about 3%, less than about 2%, less than about 1%, or 0%) of the activity of the protein produced from the full-length coding sequence of the gene. Such a portion of a gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene with inactivation thereof.

As used herein, the term “terpenoid” shall be taken to include molecules in which at least part of the molecule is derived from a prenyl pyrophosphate, such as IPP, DMAPP, etc.

It is noted that the terms “pyrophosphate” and “diphosphate” are used interchangeably herein.

As used herein, the term “cisoid” refers to terpenes, terpenoids, and precursors thereof that were derived from (2Z,6E)-FPP or a derivative thereof.

As used herein, the term “transoid” refers to terpenes, terpenoids, and precursors thereof that were derived from (2E,6E)-FPP or a derivative thereof.

As used herein, the term “metabolite” refers to byproducts of production of (2Z,6E)-FPP and/or (2E,6E)-FPP. These metabolites include but are not limited to: (2Z,6E)-farnesol and (2Z,6E)-nerolidol, derived from production of (2Z,6E)-FPP; and (2E,6E)-farnesol and (2E,6E)-nerolidol, derived from production of (2E,6E)-FPP.

Regarding sequence identity between nucleotide and amino acid sequences as set forth herein, and as would be understood by the skilled worker, a high level of sequence identity indicates likelihood that a first sequence is derived from a second sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity according to the present invention is determined by aid of computer analysis, such as, without limitations, the ClustalW computer alignment program (Higgins et al., 1994, Nucleic Acids Res. 22: 4673-4680), and the default parameters suggested therein. The ClustalW software is available from as a ClustalW WWW Service at the European Bioinformatics Institute http://www.ebi.ac.uk/clustalw. Using this program with its default settings, the mature (bioactive) part of a query and a reference polypeptide are aligned. The number of fully conserved residues are counted and divided by the length of the reference polypeptide. The ClustalW algorithm can similarly be used to align nucleotide sequences. Sequence identities can be calculated in a similar way as indicated for amino acid sequences. In certain embodiments, the cell of the present invention comprises a nucleic acid sequence encoding modified, heterologous and additional enzymatic components of terpene and terpenoid biosynthetic pathways, as defined herein.

In one aspect, the invention relates to a method for producing a terpene, terpenoid, or precursor thereof in a recombinant host cell, the method comprising the steps of culturing said recombinant host cell under conditions wherein the terpene or terpenoid is produced in a genetically engineered cell having reduced expression of endogenous FPPS, GPPS or an enzyme having both FPPS and GPPS activity, and further comprising one or more recombinant expression constructs encoding heterologous enzymes for producing said terpene, terpenoid, or precursor thereof.

In another aspect, the invention relates to a method for producing a terpene, terpenoid, or precursor thereof in a recombinant host cell, the method comprising the steps of culturing said recombinant host cell under conditions wherein the terpene or terpenoid is produced in a genetically engineered cell having reduced expression of FPPS, GPPS or an enzyme having both FPPS and GPPS activity, wherein the level of expression is optimized such that the recombinant host cell accumulates IPP, DMAPP, and GPP while still producing enough (2E,6E)-FPP to maintain normal membrane biogenesis, and further comprising one or more recombinant expression constructs encoding heterologous enzymes for producing said terpene, terpenoid, or precursor thereof.

In another aspect, the invention relates to a method for producing a cisoid terpene, terpenoid, or precursor thereof in a recombinant host cell, the method comprising the steps of culturing said recombinant host cell under conditions wherein (2Z,6E)-farnesyl diphosphate (FPP) is produced in a genetically engineered cell having reduced expression of FPPS, GPPS or an enzyme having both FPPS and GPPS activity, wherein the level of expression is optimized such that the recombinant host cell accumulates IPP, DMAPP, and GPP while still producing enough (2E,6E)-FPP to maintain normal membrane biogenesis, and further comprising one or more recombinant expression constructs encoding heterologous enzymes for producing said cisoid terpene, terpenoid, or precursor thereof.

The methods of the invention can be used, for example, for large-scale production of a terpene and/or a terpenoid by a recombinant host cell, as described for the methods of the invention. As shown in the examples that follow, the methods of the invention can be used to produce recombinant host cells with increased metabolic flux through the pathway of interest and efficient production of a terpene and/or a terpenoid of interest or a precursor thereof at unexpectedly higher levels in a recombinant host cell.

Downregulation of (2E,6E)-Farnesyl Diphosphate Synthase and/or Geranyl Diphosphate Synthase

In one aspect, the invention relates to host cells having reduced activity or expression of endogenous (2E,6E)-farnesyl diphosphate synthase ((2E,6E)-FPPS) and/or geranyl diphosphate synthase (GPPS) or an enzyme having both (2E,6E)-FPPS and GPPS activity. In some embodiments, when a wild type host cell expresses an enzyme with both (2E,6E)-FPPS and GPPS activity, then the host cells of the invention preferably have reduced activity of said enzyme with both (2E,6E)-FPPS and GPPS activity. A non-limiting example of this is the host cell is S. cerevisiae and the endogenous enzyme encoded by the ERG20 gene.

In some embodiments of the invention, the wild type host cells do not express any enzyme with both (2E,6E)-FPPS and GPPS activity. In such an embodiment, the host cells preferably have reduced activity of (2E,6E)-FPPS and/or GPPS.

Said reduced activity results in production or accumulation or both of IPP and DMAPP and thus the host cells of the invention are useful in methods for accumulating and producing IPP, DMAPP as well as compounds having IPP or DMAPP as precursors, and for producing increased amounts of terpenes or terpenoids produced from said isoprenoid precursors.

The (2E,6E)-FPPS can be any of the farnesyl pyrophosphate synthases described herein. In general the host cell carries an endogenous gene encoding (2E,6E)-FPPS, where the recombinant cell as provided by the invention has been genetically engineered in order to reduce the activity of (2E,6E)-FPPS.

The GPPS can be any of the geranyl pyrophosphate synthases described herein. In general the recombinant cell as provided by the invention has been genetically engineered in order to reduce the activity of GPPS.

Some host cells comprise a GPPS which also has some GGPP synthase activity. In embodiments of the invention using such host cells, then the GPPS can be an enzyme having both GPPS and GGPP synthase activity

When the host cell carries an endogenous gene encoding an enzyme with both (2E,6E)-FPPS and GPPS activity, then the recombinant cell as provided by the invention has been genetically engineered to reduce the activity of said enzyme.

A recombinant cell having reduced activity of (2E,6E)-FPPS activity according to the invention can have an activity of (2E,6E)-FPPS, which is about 80%, about 50%, about 30%, for example in the range of 10 to 50% of the activity of (2E,6E)-FPPS in a similar cell having wild type (2E,6E)-FPPS activity. It is in general important that the recombinant cell retains at least some (2E,6E)-FPPS activity, since this is essential for most cells. As shown herein, (2E,6E)-FPPS activity can be greatly reduced without significantly impairing cell viability. Recombinant cells with greatly reduced (2E,6E)-FPPS activity can have a somewhat slower growth rate than corresponding wild type cells. Thus it is preferred that recombinant cells of the invention have a growth rate which is at least 50% of the growth of a similar cell having wild type (2E,6E)-FPPS activity.

In certain embodiments of the invention the host cell having reduced activity of an enzyme with both (2E,6E)-FPPS and GPPS activity according to the invention has an activity of said enzyme, which is at the most 80%, preferably at the most 50%, such as at the most 30%, for example in the range of 10 to 50% of the activity of said enzyme in a similar host cell having a wild type enzyme with both (2E,6E)-FPPS and GPPS activity. It is in general important that recombinant cells retain at least some (2E,6E)-FPPS activity and at least some GPPS activity, since this is essential for most host cells. As shown herein, both the (2E,6E)-FPPS and GPPS activity can be greatly reduced without significantly impairing cell viability. Recombinant cells with greatly reduced activity can have a somewhat slower growth rate than corresponding wild type cells. Thus it is preferred that the recombinant cells of the invention have a growth rate which is at least 50% of the growth of a similar cell having a wild enzyme with both (2E,6E)-FPPS and GPPS activity.

In other embodiments of the invention, recombinant cells having reduced activity of GPPS activity according to the invention has an activity of GPPS, which is at the most 80%, preferably at the most 50%, such as at the most 30%, for example in the range of 10 to 50% of the activity of GPPS in a similar host cell having wild type GPPS activity. It is in general important that the recombinant cell retains at least some GPPS activity, since this is essential for most host cells. As shown herein, GPPS activity can be greatly reduced without significantly impairing viability. Recombinant cells with greatly reduced GPPS activity can have a somewhat slower growth rate than corresponding wild type cells. However, it is preferred that recombinant cells of the invention have a growth rate which is at least 50% of the growth of a similar host cell having wild type GPPS activity.

The activity of (2E,6E)-FPPS can be reduced in a number of different ways. In certain embodiments, the wild type promoter of a gene encoding (2E,6E)-FPPS can be exchanged for a weak promoter, such as any of the weak promoters described herein below in the section “Promoter sequence”. The endogenous gene can therefore be inactivated by introduction of a construct including a weak promoter, either by homologous recombination or by deletion and insertion. Accordingly, the recombinant cell can comprise an ORF encoding (2E,6E)-FPPS under the control of a weak promoter, which for example can be any of the weak promoters described in the section “Promoter sequence”. In general, cells of the invention only contain one ORF encoding the (2E,6E)-FPPS endogenous to the host cell, ensuring that the overall level of the endogenous (2E,6E)-FPPS activity is reduced.

In other embodiments, alternatively or simultaneously, the recombinant cell can comprise a heterologous insert sequence, which reduces the expression of mRNA encoding (2E,6E)-FPPS. In some embodiments, the heterologous nucleic acid insert sequence can be positioned between the promoter sequence and the ORF encoding (2E,6E)-FPPS. Said heterologous insert sequence can be any of the heterologous insert sequences described herein below in the section “Heterologous insert sequence”.

In further embodiments, (2E,6E)-FPPS activity can be reduced using a motif that de-stabilizes mRNA transcripts. Thus, recombinant cells of this invention can comprise a nucleic acid comprising a promoter sequence operably linked to an open reading frame (ORF) encoding (2E,6E)-FPPS, and a nucleotide sequence comprising a motif that de-stabilizes mRNA transcripts. Said motif can be any of the motif that de-stabilize mRNA transcripts described herein below in the section “Motif that de-stabilize mRNA transcripts”.

Similarly, the activity of an enzyme with both (2E,6E)-FPPS and GPPS activity or an enzyme with GPPS activity can be reduced using the same or similar methods.

In some embodiments of the invention, the recombinant cell can also have inactivated and/or no endogenous (2E,6E)-FPPS activity and/or no endogenous GPPS activity. This can for example be accomplished by:
a) deletion of the entire gene encoding endogenous (2E,6E)-FPPS; or
b) deletion of the entire coding region encoding endogenous (2E,6E)-FPPS; or
c) deletion of part of the gene encoding (2E,6E)-FPPS leading to a total loss of endogenous (2E,6E)-FPPS activity; or
d) deletion of the entire gene encoding endogenous GPPS; or
e) deletion of the entire coding region encoding endogenous GPPS; or
f) deletion of part of the gene encoding endogenous GPPS leading to a total loss of (2E,6E)-FPPS activity; or
g) deletion of the entire gene encoding an endogenous enzyme with both (2E,6E)-FPPS and GPPS activity; or
h) deletion of the entire coding region encoding an endogenous enzyme with both (2E,6E)-FPPS and GPPS activity; or
i) deletion of part of the gene encoding an endogenous enzyme with both (2E,6E)-FPPS and GPPS activity leading to a total loss of activity of said enzyme.

(2E,6E)-FPPS activity and geranyl synthase activity are generally essential for host cells, since FPP and GPP are precursors for essential cellular constituents, e.g. ergosterol. Accordingly, in embodiments of the invention where the host cell or recombinant cell have no endogenous (2E,6E)-FPPS activity:
a) cells are cultivated in the presence of ergosterol; or
b) cells comprise a heterologous nucleic acid encoding an enzyme with (2E,6E)-FPPS activity.

Similarly, in embodiments of the invention where the host cell or recombinant have no endogenous GPPS activity, in advantageous embodiments:
a) cells are cultivated in the presence of ergosterol; or
b) cells comprise a heterologous nucleic acid encoding an enzyme with GPPS and (2E,6E)-FPPS activity.

In another aspect, the invention provides recombinant cells for producing a terpene or terpenoid that are genetically engineered to have reduced expression of endogenous (2E,6E)-FPPS, GPPS or an enzyme having both (2E,6E)-FPPS and GPPS activity, and further comprising one or more recombinant expression constructs encoding heterologous enzymes for producing said terpene or terpenoid.

### Host and Recombinant Cells

Host and recombinant cells provided herein can be any cell suitable for protein expression (i.e., expression of heterologous genes) including, but not limited to, eukaryotic cells, prokaryotic cells, yeast cells, fungal cells, mammalian cells, plant cells, microbial cells and bacterial cells. Furthermore, cells according to the invention meet one or more of the following criteria: said cells should be able grow rapidly in large fermenters, should produce small organic molecules in an efficient way, should be safe and, in case of pharmaceutical embodiments, should produce and modify the products to be as similar to “human” as possible. Furthermore, a host cell is a cell that can be genetically engineered according to the invention to produce a recombinant cell, which is a cell wherein a nucleic acid has been disabled (by deletion or otherwise), or substituted (for example, by homologous recombination at a genetic locus to change the phenotype of the cell, inter alia, to produce reduced expression of a cellular enzyme or any gene of interest), or a heterologous nucleic acid, inter alia, encoding an enzyme or enzymes to confer a novel or enhanced phenotype on the cell has been introduced.

In some embodiments, recombinant cells are yeast cells that are of yeast species Saccharomyces cerevisiae, Schizosaccharomyces pombe, Yarrowia lipolytica, Candida glabrata, Ashbya gossypii, Cyberlindnera jadinii, Candida albicans, Arxula adeninivorans, Candida boidinii, Hansenula polymorpha, Kluyveromyces lacti and Pichia pastoris. Yeasts are known in the art to be useful as host cells for genetic engineering and recombinant protein expression. Yeast of different species differ in productivity and with respect to their capabilities to process and modify proteins and to secrete metabolic products thereof. The different ‘platforms’ of types of yeast make them better suited for different industrial applications. In general, yeasts and fungi are excellent host cells to be used with the present invention. They offer a desired ease of genetic manipulation and rapid growth to high cell densities on inexpensive media. As eukaryotes, they are able to perform protein modifications like glycosylation (addition of sugars), thus producing even complex foreign proteins that are identical or very similar to native products from plant or mammalian sources.

In other embodiments, the host cell for genetic engineering as set forth herein is a microalgal cell such as a cell from Chiorella or Prototheca species. In other embodiment, the host cell is a cell of a filamentous fungus, for example Aspergillus species. In other embodiments, the host cell is a plant cell. In yet additional embodiments, the host cell is a mammalian cell, such as a human, feline, porcine, simian, canine, murine, rat, mouse or rabbit cell. The host cell can also be a CHO, CHO-K1, HEI193T, HEK293, COS, PC12, HiB5, RN33b, BHK cell. In other embodiments, the host cell can be a prokaryotic cell, such as a bacterial cell, including, but not limited to E. coli or cells of Corynebacterium, Bacillus, Pseudomonas and Streptomyces species.

In certain embodiments, the host cell is a cell that, in its nonrecombinant form comprises a gene encoding at least one of the following:
(2E,6E)-farnesyl diphosphate synthase ((2E,6E)-FPPS)
geranyl diphosphate synthase (GPPS)
an enzyme having both (2E,6E)-FPPS and GPPS activity

In other embodiments, the host cell is a cell that in its nonrecombinant form comprises a gene encoding an enzyme having both (2E,6E)-FPPS and GPPS activity. For example, the host cell can be S. cerevisiae that comprises non-recombinant, endogenous ERG20, and which according to this invention can be recombinantly manipulated for reduced expression of the ERG20 gene.

### Heterologous Insert Sequence

In some embodiments the recombinant cells of the invention comprise a heterologous nucleic acid insert sequence positioned between the promoter sequence and the ORF encoding (2E,6E)-FPPS, GPPS, or an enzyme having both (2E,6E)-FPPS and GPPS activities. In these embodiments of the invention the promoter can be any promoter directing expression of said ORF in the host cell, such as any of the promoters described herein in the section “Promoter sequence”. Thus, the promoter can be a weak promoter wherein the promoter activity is less than the promoter activity of the wild type promoter in strength. In a non-limiting example, said weak promoter has decreased promoter activity compared to the ERG20 promoter in S. cerevisiae. Thus, in embodiments of the invention wherein the nucleic acid comprises a heterologous nucleic acid insert sequence between the promoter sequence and the ORF encoding (2E,6E)-FPPS, GPPS, or an enzyme having both (2E,6E)-FPPS and GPPS activities, then the promoter sequence can be a promoter directing expression of said ORF in a wild type host cell, e.g. the wild type ERG20 promoter. The heterologous nucleic acid insert sequence can be any nucleic acid sequence that adapts the secondary structure element of a hairpin.

In some embodiments, the heterologous insert sequence can be a nucleic acid sequence having the general formula (I):

—X₁—X₂—X₃—X₄—X₅—

wherein X₂ comprises at least 4 consecutive nucleotides being complementary to, and forming a hairpin secondary structure element with at least 4 consecutive nucleotides of X₄, and wherein X₃ either comprises zero nucleotides or one or more unpaired nucleotides forming a hairpin loop between X₂ and X₄, and wherein X₄ comprises or comprises at least 4 consecutive nucleotides being complementary to, and forming a hairpin secondary structure element with at least 4 consecutive nucleotides of X₂, and wherein X₁ and X₅ comprises zero, one or more nucleotides.

X₂ and X₄ in general comprises a sequence of nucleotides. Preferably the heterologous nucleic acid insert sequence comprises sections X₂ and X₄ which are complementary and hybridizes to one another, thereby forming a hairpin. Sections X₂ and X₄ can be directly connected to each other. In other embodiments X₂ and X₄ can flank section X₃, which forms a loop—the hairpin loop. In general X₃ comprises unpaired nucleic acids.

Advantageously, the heterologous insert sequence is long enough to allow a loop to be completed, but short enough to allow a limited translation rate of the ORF following the heterologous insert sequence. In general the longer the stem of the insert stem loop sequence, the lower the translation rate. Thus, in some embodiments of the invention, where a very low translation rate of the ORF is desired, then a long heterologous insert sequence should be selected and in particular a heterologous insert sequence with long X₂ and X₄ sequences complementary to each other should be selected. Thus, in certain embodiments of the present invention the heterologous nucleic acid insert sequence comprises in the range of 10 to 50 nucleotides, preferably in the range of 10 to 30 nucleotides, more preferably in the range of 15 to 25 nucleotides, more preferably in the range of 17 to 23 nucleotides, more preferably in the range of 18 to 22 nucleotides, for example in the range of 18 to 21 nucleotides, such as 19 to 20 nucleotides.

X₂ and X₄ can individually comprise any suitable number of nucleotides, so long as a consecutive sequence of at least 4 nucleotides of X₂ is complementary to a consecutive sequence of at least 4 nucleotides of X₄. In a preferred embodiment X₂ and X₄ comprise the same number of nucleotides. It is preferred that a consecutive sequence of at least 6 nucleotides, more preferably at least 8 nucleotides, even more preferably at least 10 nucleotides, such as in the range of 8 to 20 nucleotides of X₂ is complementary to a consecutive sequence of the same amount of nucleotides of X₄.

X₂ can for example comprise in the range of 4 to 25, such as in the range of 4 to 20, for example of in the range of 4 to 15, such as in the range of 6 to 12, for example in the range of 8 to 12, such as in the range of 9 to 11 nucleotides.

X₄ can for example comprise in the range of 4 to 25, such as in the range of 4 to 20, for example of in the range of 4 to 15, such as in the range of 6 to 12, for example in the range of 8 to 12, such as in the range of 9 to 11 nucleotides.

In one preferred embodiment X₂ comprises a nucleotide sequence, which is complementary to the nucleotide sequence of X₄, i.e., it is preferred that all nucleotides of X₂ are complementary to the nucleotide sequence of X₄.

In one preferred embodiment X₄ comprises a nucleotide sequence, which is complementary to the nucleotide sequence of X₂, i.e., it is preferred that all nucleotides of X₄ are complementary to the nucleotide sequence of X₂. Very preferably, X₂ and X₄ comprises the same number of nucleotides, wherein X₂ is complementary to X₄ over the entire length of X₂ and X₄.

X₃ can be absent, i.e., X₃ can comprise zero nucleotides. It is also possible that X₃ comprises in the range of 1 to 5, such as in the range of 1 to 3 nucleotides. As mentioned above, then it is preferred that X3 does not hybridise with either X₂ or X₄.

X₁ can be absent, i.e., X₁ can comprise zero nucleotides. It is also possible that X₁ comprises in the range of 1 to 25, such as in the range of 1 to 20, for example in the range of 1 to 15, such as in the range of 1 to 10, for example in the range of 1 to 5, such as in the range of 1 to 3 nucleotides.

X₅ can be absent, i.e., X₅ can comprise zero nucleotides. It is also possible that X₅ can comprise in the range 1 to 5, such as in the range of 1 to 3 nucleotides.

The sequence can be any suitable sequence fulfilling the requirements defined herein above.

### (2E,6E)-Farnesyl Diphosphate Synthase and Geranyl Diphosphate Synthase

Recombinant cells of the invention in general comprise an open reading frame (ORF) encoding (2E,6E)-farnesyl diphosphate synthase ((2E,6E)-FPPS), geranyl diphosphate synthase (GPPS), or an enzyme having both (2E,6E)-FPPS and GPPS. Said (2E,6E)-FPPS, GPPS, or an enzyme having both (2E,6E)-FPPS and GPPS can be any (2E,6E)-FPPS, GPPS, or an enzyme having both (2E,6E)-FPPS and GPPS. Frequently it will be a (2E,6E)-FPPS, GPPS, or an enzyme having both (2E,6E)-FPPS and GPPS endogenous to the host cell. Thus, by way of example, in embodiments of the invention wherein the host cell is S. cerevisiae, then preferably the ORF encoding FPPS encodes an S. cerevisiae FPPS.

The (2E,6E)-FPPS can be any enzyme which is capable of catalysing the following chemical reaction:
GPP+IPP<=>Diphosphate+(2E,6E)-FPP

It is preferred that the (2E,6E)-FPPS according to the present invention is an enzyme categorised under EC 2.5.1.10. In some embodiments, the (2E,6E)-FPPS is a Saccharomyces cerevisiae (2E,6E)-FPPS, e.g., S. cerevisiae ERG20 (SEQ ID NO:2).

The GPPS can be any enzyme which is capable of catalysing the following chemical reaction:
DMAPP+IPP<=>Diphosphate+GPP

It is preferred that the (2E,6E)-FPPS and/or a GPPS according to the present invention is an enzyme categorised under EC 2.5.1.1. In some embodiments, the (2E,6E)-FPPS or GPPS is a Saccharomyces cerevisiae (2E,6E)-FPPS or GPPS, e.g., S. cerevisiae ERG20 (SEQ ID NO:2).

An enzyme having both (2E,6E)-FPPS and GPPS activity is capable of catalysing both of the aforementioned reactions is particularly advantageous, and that said enzyme thus is an enzyme categorised under both EC 2.5.1.1 and EC 2.5.1.10. In some embodiments, the (2E,6E)-FPPS and GPPS is a Saccharomyces cerevisiae (2E,6E)-FPPS and GPPS, e.g., S. cerevisiae ERG20 (SEQ ID NO:2).

(2E,6E)-FPPS, GPPS or an enzyme having both (2E,6E)-FPPS and GPPS activity can be from a variety of sources, such as from bacteria, fungi, plants or mammals. (2E,6E)-FPPS, GPPS or an enzyme having both (2E,6E)-FPPS and GPPS activity can be wild type embodiments thereof or a functional homologue thereof.

For example, an enzyme having both (2E,6E)-FPPS and GPPS activity can be an enzyme having both (2E,6E)-FPPS activity and GPPS activity of S. cerevisiae. Thus, said enzyme can be an enzyme of SEQ ID NO:2 or a functional homologue thereof sharing at least 70%, for example at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as at least 99.5%, such as at least 99.6%, such as at least 99.7%, such as at least 99.8%, such as at least 99.9%, such as 100% sequence identity therewith. The sequence identity is preferably calculated as described herein.

A functional homologue of an enzyme having both (2E,6E)-FPPS and GPPS activity is also capable of catalysing one or both of the following chemical reactions:
DMAPP+IPP<=>Diphosphate+GPP
   and/or
GPP+IPP<=>Diphosphate+(2E,6E)-FPP

Embodiments comprising such a homolog are advantageous as set forth further herein.

### Promoter Sequence

In certain embodiments, this invention provides recombinant host cells comprising a nucleic acid comprising a promoter sequence operably linked to an ORF encoding (2E,6E)-FPPS, GPPS, or an enzyme having both (2E,6E)-FPPS and GPPS activities, wherein said ORF preferably is endogenous to said host cell. The invention also relates to recombinant cells comprising a nucleic acid comprising a promoter sequence operably linked to an ORF, wherein said ORF encodes (2E,6E)-FPPS, GPPS, or an enzyme having both (2E,6E)-FPPS and GPPS activities. In these embodiments, a promoter sequence can be any sequence capable of directing expression of said ORF in the particular host cell.

As used herein, the term “promoter” is intended to mean a region of DNA that facilitates transcription of a particular gene. Promoters are generally located in close proximity to the genes they regulate, being encoded on the same strand as the transcribed ORF and typically upstream (towards the 5′ region of the sense strand). In order for transcription to take place, the enzyme that synthesizes RNA, known as RNA polymerase, must attach to the DNA 5′ to the beginning of the ORF. Promoters contain specific DNA sequences and response elements that provide an initial binding site for RNA polymerase and for proteins called transcription factors that recruit RNA polymerase. These transcription factors have specific activator or repressor sequences of corresponding nucleotides that attach to specific promoters and regulate gene expressions.

The promoter sequence can in general be positioned immediately adjacent to the open reading frame (ORF), or a heterologous nucleic acid insert sequence can be positioned between the promoter sequence and the ORF. Positions in the promoter are in general designated relative to the transcriptional start site, where transcription of RNA begins for a particular gene (i.e., positions upstream are negative numbers counting back from −1, for example −100 is a position 100 base pairs upstream).

The promoter sequence according to the present invention in general comprises at least a core promoter, which is the minimal portion of the promoter required to properly initiate transcription. In addition the promoter sequence can comprise one or more of the following promoter elements:
transcription start site (TSS)
a binding site for RNA polymerase
general transcription factor binding sites
proximal promoter sequence upstream of the gene that tends to contain primary regulatory elements
specific transcription factor binding sites
distal promoter sequence upstream of the gene that can contain additional regulatory elements, often with a weaker influence than the proximal promoter
binding sites for repressor proteins

### Prokaryotic Promoters

In prokaryotes, the promoter comprises two short sequences at −10 and −35 positions upstream from the transcription start site. Sigma factors not only help in enhancing RNA polymerase binding to the promoter, but also help RNAP target specific genes to transcribe. The sequence at −10 is called the Pribnow box, or the −10 element, and usually comprises the six nucleotides TATAAT. The other sequence at −35 (the −35 element) usually comprises the seven nucleotides TTGACAT. Both of the above consensus sequences, while conserved on average, are not found intact in most promoters. On average only 3 of the 6 base pairs in each consensus sequence is found in any given promoter. No naturally occurring promoters have been identified to date having an intact consensus sequences at both the −10 and −35; artificial promoters with complete conservation of the −10/−35 hexamers has been found to promote RNA chain initiation at very high efficiencies.

Some promoters also contain a UP element (consensus sequence 5′-AAAWWTWTTTTNNNAAANNN-3′; W=A or T; N=any base (SEQ ID NO:7)) centered at −50; the presence of the −35 element appears to be unimportant for transcription from the UP element-containing promoters.

### Eukaryotic Promoters

Eukaryotic promoters are also typically located upstream of the ORF and can have regulatory elements several kilobases (kb) away from the transcriptional start site. In eukaryotes, the transcriptional complex can cause the DNA to fold back on itself, which allows for placement of regulatory sequences far from the actual site of transcription. Many eukaryotic promoters contain a TATA box (sequence TATAAA), which in turn binds a TATA binding protein which assists in the formation of the RNA polymerase transcriptional complex. The TATA box typically lies very close to the transcriptional start site (often within 50 bases).

Host and recombinant cells of the present invention comprise recombinant expression constructs having a promoter sequence operably linked to a nucleic acid sequence encoding a protein including inter alia, (2E,6E)-FPPS, GPPS, or an enzyme having both (2E,6E)-FPPS and GPPS activities. The promoter sequence is not limiting for the invention and can be any promoter suitable for the host cell of choice.

In certain embodiments of the present invention the promoter is a constitutive or inducible promoter. The promoter sequence can also be a synthetic promoter.

In a further embodiment of the invention, the promoter is, in non-limiting examples, an endogenous promoter, KEX2, PGK-1, GPD1, ADH1, ADH2, PYK1, TPI1, PDC1, TEF1, TEF2, FBA1, GAL1-10, CUP1, MET2, MET14, MET25, CYC1, GAL1-S, GAL1-L, TEF1, ADH1, CAG, CMV, human UbiC, RSV, EF-1alpha, SV40, Mt1, Tet-On, Tet-Off, Mo-MLV-LTR, Mx1, progesterone, RU486 or Rapamycin-inducible promoter.

In some embodiments of the invention, the recombinant cell comprises a heterologous insert sequence between the promoter sequence and the ORF encoding (2E,6E)-FPPS, GPPS, or an enzyme having both (2E,6E)-FPPS and GPPS activities. Promoter sequences can comprise a wild type promoter, for example the promoter sequence can be the promoter directing expression of said ORF in a wild type host cell. Thus, the promoter sequence can for example be the wild type ERG20 promoter.

In some embodiments of the invention, the promoter sequence is a weak promoter. In particular, in embodiments of the invention wherein the nucleic acid does not contain a heterologous nucleic acid insert sequence, then the promoter sequence is preferably a weak promoter. A weak promoter according to the present invention is a promoter, which directs a lower level of transcription in the host cell. In particular it is preferred that the promoter sequence directs expression of an ORF encoding (2E,6E)-FPPS, GPPS, or an enzyme having both (2E,6E)-FPPS and GPPS activities at an expression level significantly lower than the expression level obtained with the wild type promoter (e.g., in yeast an ERG20 promoter). Said ORF is preferably an ORF encoding native (2E,6E)-FPPS, native GPPS, or a native enzyme having both (2E,6E)-FPPS and GPPS activities, and accordingly the ORF is preferably endogenous to the host or recombinant cell.

It can be determined whether a promoter sequence is a weak promoter or directs a lower level of transcription in the host cell, by determining the expression level of mRNA encoding (2E,6E)-FPPS, GPPS, or an enzyme having both (2E,6E)-FPPS and GPPS activities in a host cell, comprising an ORF encoding (2E,6E)-FPPS, GPPS, or an enzyme having both (2E,6E)-FPPS and GPPS activities operably linked to the potential weak promoter, and by determining the expression level of mRNA encoding (2E,6E)-FPPS, GPPS, or an enzyme having both (2E,6E)-FPPS and GPPS activities in a second reference cell comprising an ORF encoding (2E,6E)-FPPS, GPPS, or an enzyme having both (2E,6E)-FPPS and GPPS activities operably linked to the wild type ERG20 promoter. The second reference cell can be a wild type cell and preferably the tested recombinant cell is of the same species as the second cell. The expression level of mRNA encoding (2E,6E)-FPPS, GPPS, or an enzyme having both (2E,6E)-FPPS and GPPS activities can be determined using any useful method known to the skilled person such as by quantitative PCR. If the expression level of said mRNA in the host cell comprising the potential weak promoter is significantly lower than in the second reference cell, then the promoter is a weak promoter.

It is preferred that the promoter sequence to be used with the present invention directs expression of the ORF encoding (2E,6E)-FPPS, GPPS, or an enzyme having both (2E,6E)-FPPS and GPPS activities at an expression level, which is at the most 70%, such as at the most 60%, for example at the most 50%, such as at the most 40% of the expression level obtained with the wild type ERG20 promoter. The expression level is preferably determined as described above.

Thus, in certain embodiments it is preferred that the promoter sequence to be used with the present invention, when contained in a host cell and operably linked to an ORF encoding (2E,6E)-FPPS, GPPS, or an enzyme having both (2E,6E)-FPPS and GPPS activities, directs expression of said ORF in said host cell so the level of mRNA encoding (2E,6E)-FPPS in said host cell is at the most 70%, such as at the most 60%, for example at the most 50%, such as at the most 40%, preferably in the range of 10 to 50% of the level of mRNA encoding (2E,6E)-FPPS, GPPS, or an enzyme having both (2E,6E)-FPPS and GPPS activities present in a second cell containing a wild type ERG20 gene, wherein the host cell and the second cell is of the same species.

Thus, in certain embodiments it is preferred that the heterologous promoter sequence to be used with the present invention, when contained in a host cell and operably linked to an ORF encoding (2E,6E)-FPPS, GPPS, or an enzyme having both (2E,6E)-FPPS and GPPS activities, directs expression of said ORF in said host cell so the level of mRNA encoding (2E,6E)-FPPS, GPPS, or an enzyme having both (2E,6E)-FPPS and GPPS activities in said recombinant cell is at the most 70%, preferably at the most 60%, even more preferably at the most 50%, such as at the most 40%, preferably is in the range of 10 to 50% of the level of mRNA encoding (2E,6E)-FPPS, GPPS, or an enzyme having both (2E,6E)-FPPS and GPPS activities present in a second cell containing a wild type gene encoding (2E,6E)-FPPS, GPPS, or an enzyme having both (2E,6E)-FPPS and GPPS activities, wherein the recombinant cell and the second cell is of the same species.

It can also be determined whether a promoter sequence is a weak promoter or directs a lower level of transcription in the host cell, by determining the expression level of any test protein, including but not limited to a reporter gene (a non-limiting example of a reporter gene is green fluorescent protein, GFP) in a recombinant cell, comprising an ORF encoding said test protein operably linked to the potential weak promoter, and by determining the expression level of the same test protein in a second cell comprising an ORF encoding said test protein operably linked to the wild type ERG20 promoter. The second cell can be a wild type cell and preferably the tested recombinant cell is of the same species as the second cell. The expression level of test protein can be determined using any useful method known to the skilled person. For example the test protein can be a fluorescent protein and the expression level can be assessed by determining the level of fluorescence.

Thus, in a preferred embodiment of the invention the heterologous promoter sequence to be used with the present invention, when contained in a recombinant cell and operably linked to an ORF encoding a test protein, directs expression of said ORF in said recombinant cell so the level of the test protein in said recombinant cell is at the most 70%, such as at the most 60%, for example at the most 50%, such as at the most 40%, preferably in the range of 10 to 50% of the level of the test protein present in a second cell containing an ORF encoding the test protein operably linked to a wild type ERG20 promoter, wherein the host cell and the second cell is of the same species. The test protein is preferably a fluorescent protein, e.g. GFP.

Non-limiting examples of weak promoters useful with the present include the CYC-1 promoter or the KEX-2 promoter; in particular the promoter sequence can be the KEX-2 promoter. Thus in certain embodiments of the invention the heterologous promoter sequence comprises or comprises the KEX-2 promoter.

Thus, in embodiments of the invention where the ORF encodes a (2E,6E)-FPPS, then preferably said (2E,6E)-FPPS is a (2E,6E)-FPPS native to the host or recombinant cell, and the heterologous promoter sequence is a weak promoter directing expression of said native (2E,6E)-FPPS at a level, which is significantly lower than the native expression level.

In embodiments of the invention where the ORF encodes a GPPS, then preferably said GPPS is a GPPS native to the host or recombinant cell, and the heterologous promoter sequence is a weal promoter directing expression of said native GPPS at a level, which is significantly lower than the native expression level.

The term “significantly lower” as used herein preferably means at the most 70%, preferably at the most 60%, even more preferably at the most 50%, such as at the most 40%. In particular the term “significantly lower” can be used to mean in the range of 10 to 50%.

Motifs that De-Stabilize mRNA Transcripts

In certain embodiments the recombinant cells of the invention comprises a nucleic acid comprising a promoter sequence operably linked to an open reading frame (ORF) encoding (2E,6E)-FPPS, GPPS or an enzyme having both (2E,6E)-FPPS and GPPS activity, and a nucleotide sequence comprising a motif that de-stabilizes mRNA transcripts.

In this embodiment the promoter can be any of the promoters described herein in the section “Promoter sequence”, for example the promoter can be the wild type ERG20 promoter. Thus, the host cell can comprise the native (2E,6E)-FPPS gene, GPPS gene or a gene encoding an enzyme having both (2E,6E)-FPPS and GPPS activity, which has been further modified to contain, downstream of its ORF, a DNA sequence motif that reduces the half-life of the mRNA produced from this gene, such as a motif that de-stabilize mRNA transcripts. The motif that de-stabilizes mRNA transcripts can be any motif, which when positioned in the 3″-UTR of a mRNA transcript can de-stabilize the mRNA transcript and lead to reduced half-life of the transcript (see e.g. Shalgi et al., 2005 Genome Biology 6:R86). Thus, to further reduce the activity of the (2E,6E)-FPPS, GPPS or an enzyme having both (2E,6E)-FPPS and GPPS activity, a nucleotide sequence containing a motif that de-stabilizes mRNA transcripts can be introduced into the native (2E,6E)-FPPS gene, GPPS gene or a gene encoding an enzyme having both (2E,6E)-FPPS and GPPS activity, downstream of the ORF.

### Additional Heterologous Nucleic Acid

Recombinant cells of the invention can comprise one or more additional heterologous nucleic acids in addition to the nucleic acid comprising an ORF encoding (2E,6E)-FPPS and/or a GPPS operably linked to a promoter sequence. In alternative embodiments, said recombinant cells can comprise additional recombinant expression constructs that direct expression in the cell of enyzmes, inter alia, for producing terpenes or terpenoids as described herein.

In some embodiments, said heterologous nucleic acid can contain a nucleic acid encoding an enzyme useful in the biosynthesis of a compound, which is desirable to synthesize from mevalonate, for example, IPP and/or DMAPP.

The heterologous nucleic acid preferably contains a nucleic acid encoding an enzyme useful in the biosynthesis of a compound, which is desirable to synthesize from either IPP or DMAPP or from both IPP and DMAPP, for example, (2Z,6E)-FPP. Thus, the additional heterologous nucleic acid can encode an enzyme useful in the biosynthesis of a terpene, a terpenoid or an alkaloid from IPP or DMAPP.

Thus, the heterologous nucleic acid can encode any enzyme using IPP or DMAPP as a substrate. Such enzymes can be any enzyme classified under EC 2.5.1.- using IPP or DMAPP as a substrate. Examples of such enzymes include GPP synthases, FPP synthases, GGPP synthases, synthases capable of catalysing incorporation of longer isoprenoid chains (e.g. chains of up to around 10 isoprenoids) and prenyl transferases.

In particular, the heterologous nucleic acid can be selected according to the particular isoprenoid compound or terpene or terpenoid to be produced by the recombinant cell. Thus, if the recombinant cell is to be used in the production of a particular isoprenoid compound or terpene or terpenoid, then the cell can comprise one or more additional heterologous nucleic acid sequences encoding one or more enzymes of the biosynthesis pathway of that particular isoprenoid compound or terpene or terpenoid.

Thus, the heterologous nucleic acid can in certain embodiments of the invention encode a (2Z,6E)-FPPS. In particular, in embodiments of the invention wherein the recombinant cell is to be used to produce a cisoid terpene, terpenoid, or precursor thereof, then it is preferred that the recombinant cell comprises an additional heterologous nucleic acid encoding a (2Z,6E)-FPPS. Said cisoid terpene or terpenoid can for example be any of the terpenes or terpenoids described herein below in the section “Cisoid terpenes and terpenoids.” Said (2Z,6E)-FPPS can be any enzyme capable of catalyzing one or both of the following reactions:
GPP+IPP<=>(2Z,6E)-FPP
2 IPP+DMAPP<=>(2Z,6E)-FPP

In some embodiments, the (2Z,6E)-FPPS is (2Z,6E)-FPPS of SEQ ID NO: 4 or a functional homologue thereof, wherein said functional homologue shares at least 70%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity SEQ ID NO: 4. The sequence identity is preferably determined as described herein. In addition to the aforementioned sequence identity, a functional homologue of (2Z,6E)-FPPS should also be capable of catalysing above-mentioned reaction.

In some embodiments of the invention an additional heterologous nucleic acid can encode a terpene synthase. In particular, in embodiments of the invention wherein the recombinant cell is to be employed in methods for production of a cisoid terpene, then it is preferred that the recombinant cell comprises an additional heterologous nucleic acid encoding a terpene synthase that uses (2Z,6E)-FPP or a derivative thereof as a natural substrate. Said cisoid terpene can for example be any of the cisoid terpenes described herein below in the section “Methods for producing cisoid terpenoids and terpenes.”

In some embodiments of the invention an additional heterologous nucleic acid can encode a sesquiterpene synthase. In particular, in embodiments of the invention wherein the host cell is to be employed in methods for production of a cisoid sesquiterpene, then it is preferred that the host cell comprise a heterologous nucleic encoding a sesquiterpene synthase that uses (2Z,6E)-FPP or a derivative thereof as a natural substrate. Said cisoid sesquiterpene can for example be any of the cisoid sesquiterpenes described herein below in the section “Methods for producing cisoid terpenoids and terpenes.”

In some embodiments of the invention, said sesquiterpene synthase can for example be a (−)-gamma-cadinene synthase. Said (−)-gamma-cadinene synthase can be any enzyme capable of catalyzing the following reaction:
(2Z,6E)-FPP<=>(−)-gamma-cadinene

In some embodiments of the invention, said sesquiterpene synthase can for example be a 4,5-di-epi-aristolochene synthase (TEAS). Said TEAS can be any enzyme capable of catalyzing the following reactions:
(2E,6E)-FPP<=>4,5-di-epi-aristolochene
(2Z,6E)-FPP<=>cisoid terpene

In some embodiments, the TEAS is TEAS of SEQ ID NO: 6 or a functional homologue thereof, wherein said functional homologue shares at least 70%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity SEQ ID NO:8. The sequence identity is preferably determined as described herein. In addition to the aforementioned sequence identity, a functional homologue of TEAS should also be capable of catalysing above-mentioned reactions.

Recombinant cells of the invention can furthermore comprise one or more additional heterologous nucleic acids encoding one or more enzymes, for example, phosphomevalonate kinase (EC 2.7.4.2), diphosphomevalonate decarboxylase (EC 4.1.1.33), 4-hydroxy-3-methylbut-2-en-1-yl diphosphate synthase (EC 1.17.7.1), 4-hydroxy-3-methylbut-2-enyl diphosphate reductase (EC 1.17.1.2), isopentenyl-diphosphate Delta-isomerase 1 (EC 5.3.3.2), short-chain Z-isoprenyl diphosphate synthase (EC 2.5.1.68), dimethylallyltransferase (EC 2.5.1.1), geranyltranstransferase (EC 2.5.1.10) or geranylgeranyl pyrophosphate synthetase (EC 2.5.1.29).

Additionally and, in some embodiments, alternatively, recombinant cells of the invention can also comprise one or more additional heterologous nucleic acids encoding one or more enzymes, for example, acetoacetyl CoA thiolose, HMG-CoA reductase or the catalytic domain thereof, HMG-CoA synthase, mevalonate kinase, phosphomevalonate kinase, phosphomevalonate decarboxylase, isopentenyl pyrophosphate isomerase, farnesyl pyrophosphate synthase, D-1-deoxyxylulose 5-phosphate synthase, and 1-deoxy-D-xylulose 5-phosphate reductoisomerase and farnesyl pyrophosphate synthase, wherein in said alternative embodiments the cells express a phenotype of increased mevalonate production or accumulation or both.

### Methods for Producing Cisoid Terpenes or Terpenoids

As mentioned herein above, recombinant cells of this invention are useful in enhancing yield of cisoid isoprenoid pyrophosphates and/or cisoid terpenes and/or cisoid terpenoids.

Specific particular embodiments of the recombinant cells of the invention are genetically engineered in order to increase accumulation of (2Z,6E)-FPP precursors and increase yield of cisoid terpenoid or cisoid terpene products resulting from enzymatic conversion of (2Z,6E)-FPP.

Accordingly, in one aspect the invention relates to methods for producing a cisoid terpene or a cisoid terpenoid, said method comprising the steps of cultivating a recombinant cell as described herein under conditions in which a cisoid terpene or cisoid terpenoid product is produced by the cell, and isolating said cisoid terpene or terpenoid.

In one example using a recombinant yeast cell embodiment, said cell having reduced activity of the ERG20 gene results in enhanced accumulation of IPP and DMAPP. DMAPP and IPP accumulation can be exploited for increased production of (2Z,6E)-FPP when combined with a heterologous (2Z,6E)-FPPS.

The invention provides methods and recombinant cells for producing cisoid terpenes or cisoid terpenoids, particularly having increased yields thereof. In certain embodiments the cisoid terpenoid or the cisoid terpene to be produced by the methods of the invention is a hemiterpenoid, monoterpene, sesquiterpenoid, diterpenoid, sesterpene, triterpenoid, tetraterpenoid or polyterpenoid.

Recombinant cells according to the invention useful for producing said cisoid terpenes and cisoid terpenoids have been genetically engineered to exhibit reduced (2E,6E)-FPP production according to the methods set forth herein. In said embodiments, the phenotype of the recombinant cell includes decreasing turnover of IPP to (2E,6E)-FPP and/or of DMAPP to (2E,6E)-FPP. Recombinant cells according to the invention also exhibit a phenotype wherein (2Z,6E)-FPP accumulation is enhanced, by genetically engineering said cells as set forth herein. In some embodiments, the invention provides recombinant cells useful in the disclosed inventive methods for producing and recovering (2Z,6E)-FPP from said cell, wherein said recombinant cells are cultured under conditions wherein (2Z,6E)-FPP is produced by the cell, advantageously in enhanced yield.

In some embodiments, the recombinant cells further comprise, endogenously or as the result of introducing additional heterologous recombinant expression constructs, one or a plurality of enzymes comprising a metabolic pathway for producing cisoid terpenes or cisoid terpenoids according to the invention. In said embodiments, cisoid terpene or cisoid terpenoid production is enhanced as the result of reduced expression of (2E,6E)-FPP, GPP or an enzyme having both (2E,6E)-FPPS and GPPS activities, or in addition or alternatively increased accumulation of mevalonate precursors using recombinant cells and methods as set forth herein.

The invention specifically provides methods and recombinant cells for producing cisoid terpenes and cisoid terpenoids.

In some embodiments, the recombinant cells provided herein are used to produce cisoid sesquiterpenes and/or sesquiterpenoids, including but not limited to the cisoid sesquiterpenes and cisoid sesquiterpenoids described herein in the section “Cisoid terpenoids and terpenes”. As provided herein, said cisoid sesquiterpenes and/or cisoid sesquiterpenoids are produced by culturing a recombinant cell that has been genetically engineered for reduced expression of (2E,6E)-FPPS activity, GPPS activity and/or the activity of an enzyme having both (2E,6E)-FPPS and GPPS activity, and wherein said recombinant cell further comprises a recombinant expression construct encoding a heterologous (2Z,6E)-FPPS and one or more additional heterologous nucleic acids each encoding an enzyme of the biosynthetic pathway to produce said cisoid sesquiterpenoid or cisoid triterpenoid from (2Z,6E)-FPP. For example, said heterologous nucleic acids can encode any of the cisoid sesquiterpenoid or cisoid triterpenoid synthases described herein in the section “Additional heterologous nucleic acids. Exemplary sesquiterpenes and sesquiterpenoids include but are not limited to (−)-gamma-cadinene, α-cedrene, prezizaene, α-acoradiene, β-curcumene, (Z)-nerolidol, α-bisabolol, and (2Z,6E)-farnesol.

### Terpenoids and Terpenes

The invention provides methods and recombinant cells for producing cisoid terpenoids, terpenes or isoprenoids (i.e., derived from (2Z,6E)-FPP) using the recombinant cells of the invention. Said recombinant cells are characterised by reduced (2E,6E)-FPPS activity, GPPS activity and/or the activity of an enzyme having both (2E,6E)-FPPS and GPPS activity, wherein said recombinant cell further comprises a recombinant expression construct encoding a heterologous (2Z,6E)-FPPS and one or more additional heterologous nucleic acids each encoding an enzyme of the biosynthetic pathway to produce said cisoid terpenoid, terpene or isoprenoid.

Terpenoids are classified according to the number of isoprene units (depicted below) used.

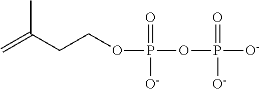

The classification thus comprises the following classes:
Hemiterpenoids, 1 isoprene unit (5 carbons)
   Examples include but are not limited to isoprene, prenol and isovaleric acid
Monoterpenoids, 2 isoprene units (10C)
   Examples include but are not limited to Geranyl pyrophosphate, Eucalyptol, Limonene and Pinene
Sesquiterpenoids, 3 isoprene units (15C)
   Examples include but are not limited to Farnesyl pyrophosphate, amorphadiene, Artemisinin and Bisabolol
Diterpenoids, 4 isoprene units (20C) (e.g. ginkgolides)
   Examples include but are not limited to Geranylgeranyl pyrophosphate, Retinol, Retinal, Phytol, Taxol, Forskolin and Aphidicolin. Another non-limiting example of a diterpene is ent-kaurene
Sesterterpenoids, 5 isoprene units (25C)
Triterpenoids, 6 isoprene units (30C)
   Examples include but are not limited to Squalene and Lanosterol
Tetraterpenoids, 8 isoprene units (40C) (e.g. carotenoids)
   Examples include but are not limited to Lycopene and Carotene and carotenoids
Polyterpenoid with a larger number of isoprene units.

Terpenes are hydrocarbons resulting from the combination of several isoprene units. Terpenoids can be thought of as terpene derivatives. The term “terpene” is sometimes used broadly to include the terpenoids. Just like terpenes, the terpenoids can be classified according to the number of isoprene units used.

The invention also relates to methods for producing other prenylated compounds. Thus the invention relates to methods for production of any compound, which has been prenylated to contain isoprenoid side-chains.

### EXAMPLES

The Examples that follow are illustrative of specific embodiments of the invention, and various uses thereof. They are set forth for explanatory purposes only, and are not to be taken as limiting the invention.

### Example 1. Engineering of (2E,6E)-FPP Production-Minimized S. cerevisiae Strain

A S. cerevisiae strain expressing a gene encoding an endogenous (2E,6E)-FPPS polypeptide (SEQ ID NO:1, SEQ ID NO:2) was engineered to minimize (2E,6E)-FPP production and accumulate IPP, DMAPP, and GPP. ERG20 expression was downregulated with a weak promoter, KEX2 (SEQ ID NO:8), to a level that allows the host to maintain normal membrane biogenesis.

### Example 2. Engineering of Cisoid Terpene-Producing S. cerevisiae Strain

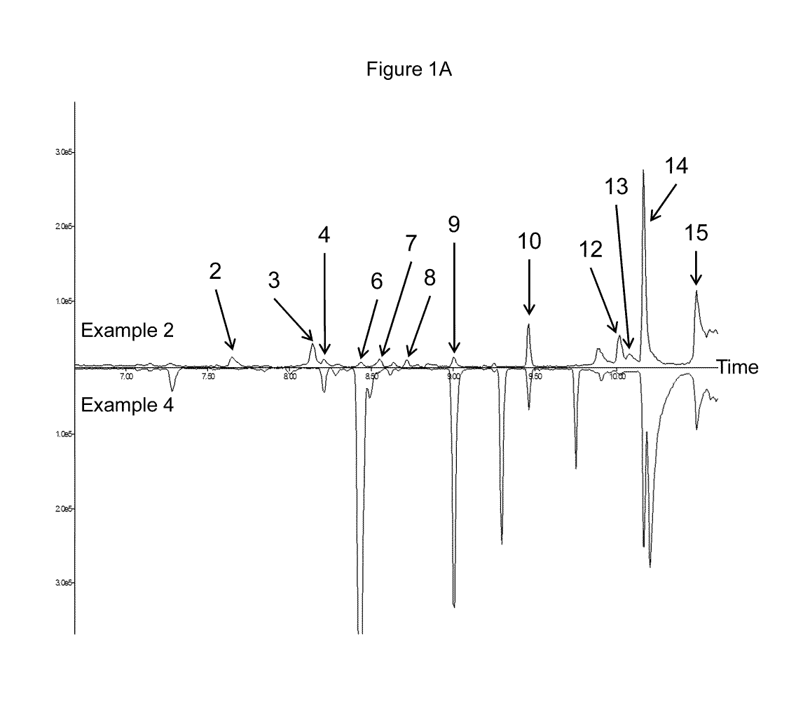

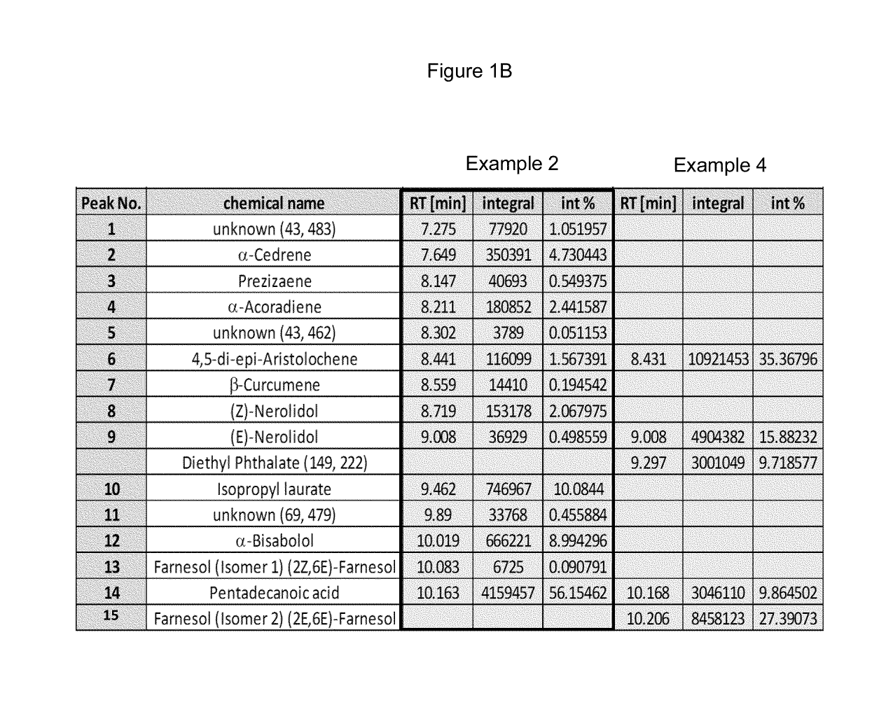

The S. cerevisiae strain of Example 1 was transformed with plasmids containing a gene encoding a heterologous (2Z,6E)-FPPS polypeptide (SEQ ID NO:3, SEQ ID NO:4) and a gene encoding a heterologous TEAS polypeptide (SEQ ID NO:5, SEQ ID NO:6). Transformants were grown in glucose media in shake flasks for 72 hours at 30° C. with 10% v/v isopropyl myristate layer. The isopropyl myristate phase trapped terpenes produced by the strain and was analyzed by gas chromatography/mass spectrometry (GC/MS) after culturing. The expressed heterologous (2Z,6E)-FPPS polypeptide (SEQ ID NO:3, SEQ ID NO:4) catalyzed production of (2Z,6E)-FPP from the accumulated IPP and DMAPP. Expression of the heterologous TEAS polypeptide (SEQ ID NO:5, SEQ ID NO:6) in this strain resulted in production of cisoid terpenes, as compared to Example 4 (below and in FIG. 1A-B). As shown in FIG. 1A-B, cisoid terpenes including, but not limited to, α-cedrene, prezizaene, α-acoradiene, β-curcumene, (Z)-Nerolidol, α-bisabolol, and (2Z,6E)-farnesol accumulated upon expression of the (2Z,6E)-FPPS and TEAS genes in the transformed S. cerevisiae strain.

### Example 3. Engineering of (2E,6E)-FPP Production-Optimized S. cerevisiae Strain

A S. cerevisiae strain expressing a gene encoding an endogenous (2E,6E)-FPPS polypeptide (SEQ ID NO:1, SEQ ID NO:2) was engineered to accumulate (2E,6E)-FPP. The expression of ERG9, which would normally catalyze the conversion of (2E,6E)-FPP to squalene, was downregulated with a CYC1 promoter (SEQ ID NO:9), which also adds a stemloop on the ERG9 transcript, slowing down translation of ERG9. In turn, this resulted in accumulation of (2E,6E)-FPP, rather than conversion to squalene.

### Example 4. Engineering of Transoid Terpene-Producing S. cerevisiae Strain

The optimized S. cerevisiae strain of Example 3 was transformed with a plasmid expressing a heterologous TEAS polypeptide (SEQ ID NO:5, SEQ ID NO:6). Transformants were grown in glucose media in shake flasks for 72 hours at 30° C. with 10% v/v isopropyl myristate layer. Terpenes produced by the engineered strain were trapped in the isopropyl myristate phase, which was analyzed by gas chromatography/mass spectrometry (GC/MS) after culturing. Expression of the heterologous TEAS polypeptide (SEQ ID NO:5, SEQ ID NO:6) in this strain resulted in exploiting the accumulated (2E,6E)-FPP to produce transoid terpenes, as compared to Example 2 (above and in FIG. 1A-B). As shown in FIG. 1A-B, the results show that only transoid terpenes, including 4,5-di-epi-aristolochene, (E)-Nerolidol, and (2E,6E)-farnesol, accumulated upon expression of the TEAS gene in the transformed S. cerevisiae strain.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

### SEQUENCES

TABLE 1
Nucleic acid and amino acid sequences.

| SEQ ID NO | Description |
|---|---|
| SEQ ID NO: 1 | Nucleotide sequence of (2E,6E)-FPPS (ERG20 gene) |
| | from S. cerevisiae |
| SEQ ID NO: 2 | Protein sequence of (2E,6E)-FPPS (ERG20 gene) from |
| | S. cerevisiae |
| SEQ ID NO: 3 | Nucleotide sequence of (2Z,6E)-FPPS (Rv1086 gene) |
| | from M. tuberculosis |
| SEQ ID NO: 4 | Protein sequence of (2Z,6E)-FPPS (Rv1086 gene) from |
| | M. tuberculosis |
| SEQ ID NO: 5 | Nucleotide sequence of 4,5-di-epi-aristolochene synthase |
| | (TEAS gene) from N. attenuata |
| SEQ ID NO: 6 | Protein sequence of 4,5-di-epi-aristolochene synthase |
| | (TEAS gene) from N. attenuata |
| SEQ ID NO: 7 | UP promoter element |
| SEQ ID NO: 8 | KEX2 promoter sequence |
| SEQ ID NO: 9 | CYC1 promoter sequence |

TABLE 2
Sequences disclosed in Table 1.

| SEQ ID NO | Sequence |
|---|---|
| SEQ ID NO: 1 | ATGGCTTCAGAAAAAGAAATTAGGAGAGAGAGATTCTTGAACGTTTTCCCTAAATTAGTAGAGG |
| | AATTGAACGCATCGCTTTTGGCTTACGGTATGCCTAAGGAAGCATGTGACTGGTATGCCCACTC |
| | ATTGAACTACAACACTCCAGGCGGTAAGCTAAATAGAGGTTTGTCCGTTGTGGACACGTATGCT |
| | ATTCTCTCCAACAAGACCGTTGAACAATTGGGGCAAGAAGAATACGAAAAGGTTGCCATTCTAG |
| | GTTGGTGCATTGAGTTGTTGCAGGCTTACTTCTTGGTCGCCGATGATATGATGGACAAGTCCAT |
| | TACCAGAAGAGGCCAACCATGTTGGTACAAGGTTCCTGAAGTTGGGGAAATTGCCATCAATGAC |
| | GCATTCATGTTAGAGGCTGCTATCTACAAGCTTTTGAAATCTCACTTCAGAAACGAAAAATACT |
| | ACATAGATATCACCGAATTGTTCCATGAGGTCACCTTCCAAACCGAATTGGGCCAATTGATGGA |
| | CTTAATCACTGCACCTGAAGACAAAGTCGACTTGAGTAAGTTCTCCCTAAAGAAGCACTCCTTC |
| | ATAGTTACTTTCAAGACTGCTTACTATTCTTTCTACTTGCCTGTCGCATTGGCCATGTACGTTG |
| | CCGGTATCACGGATGAAAAGGATTTGAAACAAGCCAGAGATGTCTTGATTCCATTGGGTGAATA |
| | CTTCCAAATTCAAGATGACTACTTAGACTGCTTCGGTACCCCAGAACAGATCGGTAAGATCGGT |
| | ACAGATATCCAAGATAACAAATGTTCTTGGGTAATCAACAAGGCATTGGAACTTGCTTCCGCAG |
| | AACAAAGAAAGACTTTAGACGAAAATTACGGTAAGAAGGACTCAGTCGCAGAAGCCAAATGCAA |
| | AAAGATTTTCAATGACTTGAAAATTGAACAGCTATACCACGAATATGAAGAGTCTATTGCCAAG |
| | GATTTGAAGGCCAAAATTTCTCAGGTCGATGAGTCTCGTGGCTTCAAAGCTGATGTCTTAACTG |
| | CGTTCTTGAACAAAGTTTACAAGAGAAGCAAATAG |
| SEQ ID NO: 2 | MASEKEIRRERFLNVFPKLVEELNASLLAYGMPKEACDWYAHSLNYNTPGGKLNRGLSVV |
| | DTYAILSNKTVEQLGQEEYEKVAILGWCIELLQAYFLVADDMMDKSITRRGQPCWYKVPE |
| | VGEIAINDAFMLEAAIYKLLKSHFRNEKYYIDITELFHEVTFQTELGQLMDLITAPEDKV |
| | DLSKFSLKKHSFIVTFKTAYYSFYLPVALAMYVAGITDEKDLKQARDVLIPLGEYFQIQD |
| | DYLDCFGTPEQIGKIGTDIQDNKCSWVINKALELASAEQRKTLDENYGKKDSVAEAKCKK |
| | IFNDLKIEQLYHEYEESIAKDLKAKISQVDESRGFKADVLTAFLNKVYKRSKMASEKEIR |
| | RERFLNVFPKLVEELNASLLAYGMPKEACDWYAHSLNYNTPGGKLNRGLSVVDTYAILSN |
| | KTVEQLGQEEYEKVAILGWCIELLQAYFLVADDMMDKSITRRGQPCWYKVPEVGEIAIND |
| | AFMLEAAIYKLLKSHFRNEKYYIDITELFHEVTFQTELGQLMDLITAPEDKVDLSKFSLK |
| | KHSFIVTFKTAYYSFYLPVALAMYVAGITDEKDLKQARDVLIPLGEYFQIQDDYLDCFGT |
| | PEQIGKIGTDIQDNKCSWVINKALELASAEQRKTLDENYGKKDSVAEAKCKKIFNDLKIE |
| | QLYHEYEESIAKDLKAKISQVDESRGFKADVLTAFLNKVYKRSK- |
| SEQ ID NO: 3 | ATGGAGATCATCCCGCCTCGGCTCAAAGAGCCGTTGTACCGGCTCTACGAGCTGCGCCTGCGGC |
| | AGGGCTTGGCCGCCTCGAAATCCGACCTGCCCCGGCACATAGCCGTGCTGTGCGACGGCAACCG |
| | GCGATGGGCGCGCAGCGCGGGCTACGACGACGTCAGCTACGGCTACCGGATGGGTGCGGCCAAG |
| | ATCGCCGAAATGCTGCGGTGGTGCCACGAAGCCGGCATCGAACTGGCCACCGTCTATCTGCTGT |
| | CCACCGAAAACCTGCAGCGCGATCCCGACGAGCTTGCAGCACTCATCGAGATCATCACCGATGT |
| | CGTGGAAGAGATCTGCGCACCGGCCAACCACTGGAGTGTGCGGACGGTCGGGGATCTGGGGTTG |
| | ATCGGCGAGGAACCGGCCCGGCGGCTGCGCGGTGCGGTGGAATCCACCCCGGAGGTGGCCTCGT |
| | TTCATGTCAACGTTGCTGTTGGCTACGGCGGGCGCCGCGAGATCGTCGACGCTGTGCGCGCGTT |
| | GTTGAGCAAGGAACTCGCCAACGGGGCCACAGCGGAGGAACTCGTCGACGCGGTGACCGTCGAG |
| | GGTATCTCGGAAAACCTGTACACCTCAGGCCAACCCGACCCCGATTTGGTGATACGCACCTCCG |
| | GCGAGCAACGCTTGTCCGGGTTCTTGCTGTGGCAAAGCGCCTACTCGGAGATGTGGTTCACCGA |
| | GGCGCACTGGCCGGCGTTTCGCCACGTCGATTTTCTACGCGCGCTGCGTGACTACAGTGCGAGG |
| | CATCGCAGCTACGGCAGGTGA |
| SEQ ID NO: 4 | MEIIPPRLKEPLYRLYELRLRQGLAASKSDLPRHIAVLCDGNRRWARSAGYDDVSYGYRM |
| | GAAKIAEMLRWCHEAGIELATVYLLSTENLQRDPDELAALIEIITDVVEEICAPANHWSV |
| | RTVGDLGLIGEEPARRLRGAVESTPEVASFHVNVAVGYGGRREIVDAVRALLSKELANGA |
| | TAEELVDAVTVEGISENLYTSGQPDPDLVIRTSGEQRLSGFLLWQSAYSEMWFTEAHWPA |
| | FRHVDFLRALRDYSARHRSYGR- |
| SEQ ID NO: 5 | ATGGCTTCTGCTGCTGTTGGTAATTATGAAGAGGAAATTGTAAGACCAGTCGCTGATTTTTCAC |
| | CTTCCTTGTGGGGAGACCATTTCTTAAGTTTTAGCATAGATAACCAAGTGGCAGAGAAATACGC |
| | CCAGGAAATCGAACCACTAAAGGAGCAAACTAGGTCTATGCTTTTGGCTACAGGCAGAAAATTA |
| | GCAGACACCCTTAATTTGATTGATACTATAGAAAGGTTGGGTATCTCTTATTACTTCGAAAAGG |
| | AGATTGACGAAATACTTGACCACATCTACAACCAGAACTCCAACTGTAACGACTTTTGCACAAG |
| | CGCCTTGCAATTCAGATTATTGAGACAACATGGCTTTAACATCTCCCCTCAGATTTTCAGCAAA |
| | TTCCAGGACGAAAATGGCAAGTTTAGGGAGTCTCTTGCTTCAGATGTTTTGGGTTTACTTAACC |
| | TATACGAGGCCTCTCACGTAAGAACCCATGCTGATGATATCTTAGAGGATGCCCTTGCATTTTC |
| | TACTATACACTTGGAAAGTGCCGCACCACACCTTAAGTCACCTCTAAGAGAACAAGTCACACAT |
| | GCACTTGAACAATGTCTACATAAGGGTGTGCCAAGGGTTGAAACCAGATTCTTCATTTCCAGCA |
| | TATATGAAAAAGAGCAAAGCAAGAATAATGTCCTTTTAAGGTTTGCTAAGTTGGACTTCAACTT |
| | ATTGCAGATGTTGCACAAACAGGAATTAGCCGAAGTATCAAGATGGTGGAAAGATCTTGATTTT |
| | GTGACCACTTTGCCTTACGCAAGAGATAGGGTTGTTGAGTGCTATTTCTGGGCTTTAGGAGTAT |
| | ACTTTGAACCACAATATTCTCAAGCCAGAGTCATGCTTGTGAAAACAATCAGCATGATTTCCAT |
| | AGTTGATGACACTTTCGATGCATACGGCACAGTAAAAGAACTAGAGGCTTATACTGACGCCATC |
| | CAAAGATGGGATATTAATGAAATTGATAGGTTGCCTCATTACATGAAAATAAGCTATAAGGCAA |
| | TTTTGGACTTATACAAGGACTACGAGAAAGAGTTGTCCAGTGCTGAAAAGTCCCATATTGTCTG |
| | CCACGCTATAGAAAGAATGAAGGAAGTTGTGGGTCATTACAACGTTGAGTCAACCTGGTTTATC |
| | GAAGGATATATGCCTCCTGTTTCCGAATACTTATCCAACGCCCTAGCAACAACTACCTACTATT |
| | ACTTGGCTACAACTAGTTATCTTGGTATGAAAAGCGCCACAGAACAAGATTTCGAGTGGTTATC |
| | AAAGAACCCAAAAATCTTGGAAGCTAGCGTCATTATATGCAGGGTGATTGATGATACCGCTACT |
| | TACGAAGTTGAGAAAAGCAGAGGCCAGATTGCCACAGGTATAGAATGTTGTATGAGAGACTATG |
| | GAATTAGCACTAAAAAGGCAATGGCCAAATTTCAGAAAATGGCAGAGACCGCTTGGAAGGATAT |
| | TAATGAAGGTTTGCTTAGGCCTACACCAGTGAGTACTGAGTTCTTGACCCTTATATTGAATCTT |
| | GCCAGAATCGTCGAGGTTACATACATTCATAATTTGGACGGCTATACTCACCCAGAAAAAGTGT |
| | TAAAGCCTCATATAATCAATCTTCTAGTCGACTCCATTAAGATCTGA |
| SEQ ID NO: 6 | MASAAVGNYEEEIVRPVADFSPSLWGDHFLSFSIDNQVAEKYAQEIEPLKEQTRSMLLAT |
| | GRKLADTLNLIDTIERLGISYYFEKEIDEILDHIYNQNSNCNDFCTSALQFRLLRQHGFN |
| | ISPQIFSKFQDENGKFRESLASDVLGLLNLYEASHVRTHADDILEDALAFSTIHLESAAP |
| | HLKSPLREQVTHALEQCLHKGVPRVETRFFISSIYEKEQSKNNVLLRFAKLDFNLLQMLH |
| | KQELAEVSRWWKDLDFVTTLPYARDRVVECYFWALGVYFEPQYSQARVMLVKTISMISIV |
| | DDTFDAYGTVKELEAYTDAIQRWDINEIDRLPHYMKISYKAILDLYKDYEKELSSAEKSH |
| | IVCHAIERMKEVVGHYNVESTWFIEGYMPPVSEYLSNALATTTYYYLATTSYLGMKSATE |
| | QDFEWLSKNPKILEASVIICRVIDDTATYEVEKSRGQIATGIECCMRDYGISTKKAMAKF |
| | QKMAETAWKDINEGLLRPTPVSTEFLTLILNLARIVEVTYIHNLDGYTHPEKVLKPHIIN |
| | LLVDSIKI- |
| SEQ ID NO: 7 | AAAWWTWTTTTNNNAAANNN |
| SEQ ID NO: 8 | TCAGCAGCTCTGATGTAGATACACGTATCTCGACATGTTTTATTTTTACTATACATACATAAAA |
| | GAAATAAAAAATGATAACGTGTATATTATTATTCATATAATCAATGAGGGTCATTTTCTGAAAC |
| | GCAAAAAACGGTAAATGGAAAAAAAATAAAGATAGAAAAAGAAAACAAACAAAGGAAAGGTTAG |
| | CATATTAAATAACTGAGCTGATACTTCAACAGCATCGCTGAAGAGAACAGTATTGAAACCGAAA |
| | CATTTTCTAAAGGCAAACAAGGTACTCCATATTTGCTGGACGTGTTCTTTCTCTCGTTTCATAT |
| | GCATAATTCTGTCATAAGCCTGTTCTTTTTCCTGGCTTAAACATCCCGTTTTGTAAAAGAGAAA |
| | TCTATTCCACATATTTCATTCATTCGGCTACCATACTAAGGATAAACTAATCCCGTTGTTTTTT |
| | GGCCTCGTCACATAATTATAAACTACTAACCCATTATCAGAAG |
| SEQ ID NO: 9 | CGTTGGTTGGTGGATCAAGCCCACGCGTAGGCAATCCTCGAGCAGATCCGCCAGGCGTGTATAT |
| | ATAGCGTGGATGGCCAGGCAACTTTAGTGCTGACACATACAGGCATATATATATGTGTGCGACG |
| | ACACATGATCATATGGCATGCATGTGCTCTGTATGTATATAAAACTCTTGTTTTCTTCTTTTCT |
| | CTAAATATTCTTTCCTTATACATTAGGACCTTTGCAGCATAAATTACTATACTTCTATAGACAC |
| | ACAAACACAAATACACACACTAAATTAATATGAATTCGTTAACGAATTCA |

What is claimed is:

1. A recombinant host comprising a gene encoding a heterologous (2Z,6E)-farnesyl diphosphate synthase ((2Z,6E)-FPPS) polypeptide;
wherein the host is capable of producing a (2Z,6E)-farnesyl diphosphate ((2Z,6E)-FPP) compound and/or a compound derived from (2Z,6E)-FPP.

2. The recombinant host of claim 1, wherein the gene encoding the (2Z,6E)-FPPS polypeptide encodes an amino acid sequence having 70% or greater identity to the amino acid sequence set forth in SEQ ID NO:4.

3. The recombinant host of claim 1 or 2, further comprising a gene encoding a terpene synthase polypeptide, wherein (2Z,6E)-FPP is a substrate for said terpene synthase.

4. The recombinant host of claim 3, wherein (2Z,6E)-FPP and (2E,6E)-farnesyl diphosphate ((2E,6E)-FPP) are substrates for said terpene synthase.

5. The recombinant host of claim 3 or 4, wherein the terpene synthase is a 4,5-di-epi-aristolochene synthase (TEAS).

6. The recombinant host of claim 5, wherein the gene encoding the TEAS polypeptide encodes an amino acid sequence having 70% or greater identity to the amino acid sequence set forth in SEQ ID NO:6.

7. A recombinant host comprising:
(a) a gene encoding a (2Z,6E)-FPPS polypeptide having 70% or greater identity to an amino acid sequence set forth in SEQ ID NO:4; and
(b) a gene encoding a 4,5-di-epi-aristolochene synthase (TEAS) polypeptide having 70% or greater identity to an amino acid sequence set forth in SEQ ID NO:6.
wherein at least one of said genes is a heterologous gene.

8. The recombinant host of any one of claims 1-7, wherein the host is engineered to have reduced expression of an endogenous gene encoding:
(a) a (2E,6E)-FPPS polypeptide;
(b) a geranyl diphosphate synthase (GPPS) polypeptide; or
(c) a polypeptide having both (2E,6E)-FPPS and GPPS enzymatic activity.

9. The recombinant host of claim 8, wherein the endogenous gene encoding a (2E,6E)-FPPS polypeptide is ERG20.

10. The recombinant host of claim 9, wherein the ERG20 gene encodes a polypeptide having 70% or greater identity to an amino acid sequence set forth in SEQ ID NO:2.

11. The recombinant host of any one of claims 1-10, wherein the host produces the compound (2Z,6E)-FPP.

12. The recombinant host of any one of claims 1-10, wherein the host produces a terpene or terpenoid compound derived from (2Z,6E)-FPP, the compound comprising α-cedrene, prezizaene, α-acoradiene, β-curcumene, (Z)-Nerolidol, α-bisabolol, and/or (2Z,6E)-farnesol.

13. The recombinant host of any one of claims 1-12 wherein the recombinant host comprises a microorganism that is a plant cell, a mammalian cell, an insect cell, a fungal cell, or a bacterial cell.

14. The recombinant host of claim 13, wherein the bacterial cell comprises Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacteria cells.

15. The recombinant host of claim 13, wherein the fungal cell comprises a yeast cell.

16. The recombinant host of claim 15, wherein the yeast cell comprises a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

17. The recombinant host of claim 16, wherein the yeast cell is a Saccharomycete.

18. The recombinant host of claim 17, wherein the yeast cell comprises a cell from the Saccharomyces cerevisiae species.

19. A method of producing (2Z,6E)-FPP comprising:
(a) growing the recombinant host of any one of claims 1-18 in a culture medium, under conditions in which the genes recited therein are expressed, wherein (2Z,6E)-FPP is synthesized by the recombinant host; and
(b) isolating (2Z,6E)-FPP.

20. The method of claim 19, wherein the (2Z,6E)-FPPS polypeptide comprises a (2Z,6E)-FPPS polypeptide having 70% or greater identity to the amino acid sequence set forth in SEQ ID NO: 4.

21. A method of producing a terpene or terpenoid derived from (2Z,6E)-FPP comprising:
(a) growing the recombinant host of any one of claim 1-10 or 12-18 in a culture medium, under conditions in which the genes discussed in any one of claim 1-10 or 12-18 are expressed, wherein the terpene or terpenoid is synthesized by the recombinant host converting (2Z,6E)-FPP to said terpene or terpenoid; and
(b) isolating the terpene or terpenoid derived from (2Z,6E)-FPP.

22. The method of claim 21, wherein the terpene or terpenoid comprises α-cedrene, prezizaene, α-acoradiene, β-curcumene, (Z)-Nerolidol, α-bisabolol, and/or (2Z,6E)-farnesol.

23. The method of claim 21 or 22, wherein the conversion of (2Z,6E)-FPP to the terpene or terpenoid is catalyzed by a terpene synthase polypeptide, wherein (2Z,6E)-FPP is a substrate for said terpene synthase.

24. The method of claim 23, wherein the terpene synthase is a 4,5-di-epi-aristolochene synthase (TEAS).

25. The method of claim 24, wherein the TEAS polypeptide encodes an amino acid sequence having 70% or greater identity to the amino acid sequence set forth in SEQ ID NO:6.

26. The method of any one of claims 21-25, further comprising a step of modifying the terpene or terpenoid.

27. The method of claim 26, wherein the terpene or terpenoid is oxygenated.

28. The method of claim 27, wherein oxygenation of the terpene or terpenoid is catalyzed by a cytochrome P450 polypeptide.

29. The method of claim 26, wherein the terpene or terpenoid is methylated.

30. The method of claim 26, wherein a sulfonate group is added to the terpene or terpenoid.

31. The method of claim 26, wherein a halogen is added to the terpene or terpenoid.

32. A cell culture broth comprising:
(a) the recombinant host of any one of claims 1-18; and
(b) (2Z,6E)-FPP, α-cedrene, prezizaene, α-acoradiene, β-curcumene, (Z)-Nerolidol, α-bisabolol, and/or (2Z,6E)-farnesol produced by the recombinant host of any one of claims 1-18;
wherein (2Z,6E)-FPP, α-cedrene, prezizaene, α-acoradiene, β-curcumene, (Z)-Nerolidol, α-bisabolol, and/or (2Z,6E)-farnesol is present at a concentration of at least 0.1 mg/liter of the culture broth.

33. The cell culture broth of claim 32, further comprising an increased level of the metabolite (2Z,6E)-farnesol relative to a cell culture broth comprising a corresponding host lacking the gene encoding a heterologous (2Z,6E)-FPPS.

34. A cell culture broth comprising (2Z,6E)-FPP, α-cedrene, prezizaene, α-acoradiene, β-curcumene, (Z)-Nerolidol, α-bisabolol, and/or (2Z,6E)-farnesol;
wherein (2Z,6E)-FPP, α-cedrene, prezizaene, α-acoradiene, β-curcumene, (Z)-Nerolidol, α-bisabolol, and/or (2Z,6E)-farnesol is present at a concentration of at least 0.1 mg/liter of the culture broth, and is produced by culturing the cells of the recombinant host of any one of claims 1-18 in a culture media.

35. A cell lysate comprising (2Z,6E)-FPP, α-cedrene, prezizaene, α-acoradiene, β-curcumene, (Z)-Nerolidol, α-bisabolol, and/or (2Z,6E)-farnesol produced by the recombinant host of any one of claims 1-18.

36. A composition of terpenes and/or terpenoids comprising (2Z,6E)-FPP, α-cedrene, prezizaene, α-acoradiene, β-curcumene, (Z)-Nerolidol, α-bisabolol, and/or (2Z,6E)-farnesol produced by the recombinant host of any one of claims 1-18, wherein the relative levels of terpenes and/or terpenoids in the composition correspond to the relative levels of terpene and/or terpenoid accumulation in the recombinant host.

37. The composition of claim 36, further comprising an increased level of the metabolite (2Z,6E)-farnesol relative to a composition of terpenes and/or terpenoids produced by a corresponding host lacking the gene encoding a heterologous (2Z,6E)-FPPS.


SEQUENCE LISTING (9 sequences)

SEQ ID NO: 1 - DNA, 1059 bp, Saccharomyces cerevisiae
atggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatag

SEQ ID NO: 2 - protein, 704 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSKMASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 3 - DNA, 789 bp, Mycobacterium tuberculosis
atggagatcatcccgcctcggctcaaagagccgttgtaccggctctacgagctgcgcctgcggcagggcttggccgcctcgaaatccgacctgccccggcacatagccgtgctgtgcgacggcaaccggcgatgggcgcgcagcgcgggctacgacgacgtcagctacggctaccggatgggtgcggccaagatcgccgaaatgctgcggtggtgccacgaagccggcatcgaactggccaccgtctatctgctgtccaccgaaaacctgcagcgcgatcccgacgagcttgcagcactcatcgagatcatcaccgatgtcgtggaagagatctgcgcaccggccaaccactggagtgtgcggacggtcggggatctggggttgatcggcgaggaaccggcccggcggctgcgcggtgcggtggaatccaccccggaggtggcctcgtttcatgtcaacgttgctgttggctacggcgggcgccgcgagatcgtcgacgctgtgcgcgcgttgttgagcaaggaactcgccaacggggccacagcggaggaactcgtcgacgcggtgaccgtcgagggtatctcggaaaacctgtacacctcaggccaacccgaccccgatttggtgatacgcacctccggcgagcaacgcttgtccgggttcttgctgtggcaaagcgcctactcggagatgtggttcaccgaggcgcactggccggcgtttcgccacgtcgattttctacgcgcgctgcgtgactacagtgcgaggcatcgcagctacggcaggtga

SEQ ID NO: 4 - protein, 262 aa, Mycobacterium tuberculosis
MEIIPPRLKEPLYRLYELRLRQGLAASKSDLPRHIAVLCDGNRRWARSAGYDDVSYGYRMGAAKIAEMLRWCHEAGIELATVYLLSTENLQRDPDELAALIEIITDVVEEICAPANHWSVRTVGDLGLIGEEPARRLRGAVESTPEVASFHVNVAVGYGGRREIVDAVRALLSKELANGATAEELVDAVTVEGISENLYTSGQPDPDLVIRTSGEQRLSGFLLWQSAYSEMWFTEAHWPAFRHVDFLRALRDYSARHRSYGR

SEQ ID NO: 5 - DNA, 1647 bp, Nicotiana attenuata
atggcttctgctgctgttggtaattatgaagaggaaattgtaagaccagtcgctgatttttcaccttccttgtggggagaccatttcttaagttttagcatagataaccaagtggcagagaaatacgcccaggaaatcgaaccactaaaggagcaaactaggtctatgcttttggctacaggcagaaaattagcagacacccttaatttgattgatactatagaaaggttgggtatctcttattacttcgaaaaggagattgacgaaatacttgaccacatctacaaccagaactccaactgtaacgacttttgcacaagcgccttgcaattcagattattgagacaacatggctttaacatctcccctcagattttcagcaaattccaggacgaaaatggcaagtttagggagtctcttgcttcagatgttttgggtttacttaacctatacgaggcctctcacgtaagaacccatgctgatgatatcttagaggatgcccttgcattttctactatacacttggaaagtgccgcaccacaccttaagtcacctctaagagaacaagtcacacatgcacttgaacaatgtctacataagggtgtgccaagggttgaaaccagattcttcatttccagcatatatgaaaaagagcaaagcaagaataatgtccttttaaggtttgctaagttggacttcaacttattgcagatgttgcacaaacaggaattagccgaagtatcaagatggtggaaagatcttgattttgtgaccactttgccttacgcaagagatagggttgttgagtgctatttctgggctttaggagtatactttgaaccacaatattctcaagccagagtcatgcttgtgaaaacaatcagcatgatttccatagttgatgacactttcgatgcatacggcacagtaaaagaactagaggcttatactgacgccatccaaagatgggatattaatgaaattgataggttgcctcattacatgaaaataagctataaggcaattttggacttatacaaggactacgagaaagagttgtccagtgctgaaaagtcccatattgtctgccacgctatagaaagaatgaaggaagttgtgggtcattacaacgttgagtcaacctggtttatcgaaggatatatgcctcctgtttccgaatacttatccaacgccctagcaacaactacctactattacttggctacaactagttatcttggtatgaaaagcgccacagaacaagatttcgagtggttatcaaagaacccaaaaatcttggaagctagcgtcattatatgcagggtgattgatgataccgctacttacgaagttgagaaaagcagaggccagattgccacaggtatagaatgttgtatgagagactatggaattagcactaaaaaggcaatggccaaatttcagaaaatggcagagaccgcttggaaggatattaatgaaggtttgcttaggcctacaccagtgagtactgagttcttgacccttatattgaatcttgccagaatcgtcgaggttacatacattcataatttggacggctatactcacccagaaaaagtgttaaagcctcatataatcaatcttctagtcgactccattaagatctga

SEQ ID NO: 6 - protein, 548 aa, Nicotiana attenuata
MASAAVGNYEEEIVRPVADFSPSLWGDHFLSFSIDNQVAEKYAQEIEPLKEQTRSMLLATGRKLADTLNLIDTIERLGISYYFEKEIDEILDHIYNQNSNCNDFCTSALQFRLLRQHGFNISPQIFSKFQDENGKFRESLASDVLGLLNLYEASHVRTHADDILEDALAFSTIHLESAAPHLKSPLREQVTHALEQCLHKGVPRVETRFFISSIYEKEQSKNNVLLRFAKLDFNLLQMLHKQELAEVSRWWKDLDFVTTLPYARDRVVECYFWALGVYFEPQYSQARVMLVKTISMISIVDDTFDAYGTVKELEAYTDAIQRWDINEIDRLPHYMKISYKAILDLYKDYEKELSSAEKSHIVCHAIERMKEVVGHYNVESTWFIEGYMPPVSEYLSNALATTTYYYLATTSYLGMKSATEQDFEWLSKNPKILEASVIICRVIDDTATYEVEKSRGQIATGIECCMRDYGISTKKAMAKFQKMAETAWKDINEGLLRPTPVSTEFLTLILNLARIVEVTYIHNLDGYTHPEKVLKPHIINLLVDSIKI

SEQ ID NO: 7 - DNA, 20 bp, Artificial Sequence
  UP element
  misc_feature (4)..(5)
  misc_feature (4)..(5): w is a or t
  misc_feature (7)..(7): w is a or t
  misc_feature (12)..(14): n is a, c, g, or t
  misc_feature (18)..(20): n is a, c, g, or t
aaawwtwttttnnnaaannn

SEQ ID NO: 8 - DNA, 491 bp, Artificial Sequence
  KEX2 promoter
tcagcagctctgatgtagatacacgtatctcgacatgttttatttttactatacatacataaaagaaataaaaaatgataacgtgtatattattattcatataatcaatgagggtcattttctgaaacgcaaaaaacggtaaatggaaaaaaaataaagatagaaaaagaaaacaaacaaaggaaaggttagcatattaaataactgagctgatacttcaacagcatcgctgaagagaacagtattgaaaccgaaacattttctaaaggcaaacaaggtactccatatttgctggacgtgttctttctctcgtttcatatgcataattctgtcataagcctgttctttttcctggcttaaacatcccgttttgtaaaagagaaatctattccacatatttcattcattcggctaccatactaaggataaactaatcccgttgttttttggcctcgtcacataattataaactactaacccattatcagaag

SEQ ID NO: 9 - DNA, 306 bp, Artificial Sequence
  CYC1 promoter with stemloop sequence
cgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaatatgaattcgttaacgaattca